(19) United States
(12) Patent Application Publication
(10) Pub. No.: US 2026/0334519 A1
(43) Pub. Date: Oct. 29, 2026

(54) VITAMIN A-FREE NUTRITIONAL COMPOSITIONS FOR TREATING DYSFUNCTION OF LIVER FAT METABOLISM

(71) Applicant: LEIBNIZ-INSTITUT FÜR ALTERNSFORSCHUNG - FRITZ-LIPMANN-INSTITUT E.V. (FLI), Jena (DE)

(72) Inventors: Karl Lenhard RUDOLPH, Jena (DE); Stefan TÜMPEL, Jena (DE); Friedrich BECKER, Jena (DE)

(73) Assignee: LEIBNIZ-INSTITUT FÜR ALTERNSFORSCHUNG - FRITZ-LIPMANN-INSTITUT E.V. (FLI), Jena (DE)

(21) Appl. No.: 19/099,813

(22) PCT Filed: Aug. 2, 2023
(86) PCT No.: PCT/EP2023/071429

§ 371 Date: Jan. 30, 2025

(30) Foreign Application Priority Data
Aug. 2, 2022 (EP) 22188270.7

(51) Int. Cl. A23L 33/00

(52) U.S. Cl. CPC A23L 33/30

(57) ABSTRACT

The invention relates to a composition for use as a dietary treatment in preventing and/or treating an impairment and/or dysfunction of liver fat metabolism and/or a medical condition associated therewith, wherein the composition comprises dietary nutrients and is substantially free of vitamin A and/or derivatives thereof. In embodiments, the processed food product comprises an amount of dietary nutrients covering the daily requirements, or a part thereof, of a subject. The invention further relates to a method for reducing the vitamin A intake of a subject without substantially reducing retinol serum levels and/or retinol levels in the eye of the subject, comprising replacing the diet of the subject or parts thereof by said composition.

The invention is in the field of compositions, preferably dietary compositions, for use in the prevention and/or treatment of an impairment and/or dysfunction of liver fat metabolism or a medical condition associated therewith.

Medical conditions associated with an impairment and/or dysfunction of liver fat metabolism include, for example:
(i) a muscle disease, such as a loss of muscle mass and/or loss of strength in a subject, such as occurring during aging, in response to a diet, in response to immobilization, and/or in response to a liver disease associated with reduced liver fat metabolism, such as cirrhosis, nonalcoholic steatohepatitis (NASH) and nonalcoholic fatty liver disease (NAFLD),
(ii) a liver disease that leads to impairments and/or dysfunction in fat metabolism, such as cirrhosis, NASH, NAFLD and liver aging, and/or
(iii) a metabolic disease such as metabolic syndrome, diabetes mellitus, insulin resistance and/or or elevated blood levels of triglycerides, LDL (low density lipoprotein, also termed LDL-cholesterin), cholesterin and/or total lipids that is induced and/or aggravated by impairments and/or dysfunction in liver fat metabolism.

The invention relates to a composition for use as a dietary treatment in preventing and/or treating an impairment and/or dysfunction in liver fat metabolism, wherein the composition comprises dietary nutrients and is substantially free of vitamin A and/or derivatives thereof (herein referred to as vitamin A free diet or “VAFD”). In embodiments, composition comprises an amount of dietary nutrients covering the daily requirements, or a part thereof, of a subject. In embodiments the composition comprises a processed food product.

The invention further relates to a method for reducing the vitamin A intake of a subject without substantially reducing retinol serum levels or retinol levels in the eye of the subject, comprising replacing the diet of the subject or parts thereof by a composition comprising dietary nutrients and that is substantially free of vitamin A and/or derivatives thereof.

### BACKGROUND OF THE INVENTION

Impairment in liver fat metabolism represents a hallmark feature of several associated diseases, such as liver diseases including cirrhosis (Dhaliwei et al., 2020), non-alcoholic fatty liver disease (NAFLD), and non-alcoholic steatohepatitis (NASH). Impairments in liver fat metabolism lead to functional compromise of the liver as the main metabolic functions of the liver employ fat metabolism derived Acetyl COA to generate ATP (using it as a caron source for the TCA cycle) or for gluconeogenesis (Pietrocola et al. 2015). Impairments in liver fat metabolism thus contribute to failure in liver function in such diseases. Moreover, the liver will then activate catabolic processes in peripheral organs to replace the loss of hepatic fat metabolism in providing Acetyl CoA to maintain major metabolic functions of the liver (see aforementioned examples, Lee et al., 2016).

Impairments in liver fat metabolism further promote the induction of systemic defects in metabolism such as insulin resistance, metabolic syndrome, and diabetes mellitus (Cariou 2022) thereby impacting on the development of cardiovascular diseases and neurocognitive dysfunction (Rinaldi et al., 2021). Improvements in lipid metabolism of the liver represent a promising target to prevent such metabolic diseases. A powerful dietary intervention that could achieve this goal is currently not available.

Further, an impairment of liver fat metabolism is associated with muscle related medical conditions and diseases such as a loss of muscle mass and strength, and sarcopenia. Loss of muscle mass and function are a universal symptom of advanced aging, as age-related loss of muscle mass and strength starts between the ages of 30 and 40. With increasing age, this process accelerates and, if more pronounced, is referred to as sarcopenia (Rosenberg et al., 1997; Cruz-Jentoft et al., 2019). Loss of muscle mass and strength profoundly impairs the participation in physical activities, induces a progressive reduction in the quality of life, increases the risk of injury and metabolic disease, and associates with increased mortality. The incidence of sarcopenia in old age is 10% in >60-year-olds (Cruz-Jentoft et al., 2019). Mechanistically, the development of sarcopenia involves a combination of muscle cell intrinsic processes, such as alterations in protein homeostasis (e.g., increases in proteolysis, impaired maintenance of structural components of muscle contraction, such as sarcomeres) and muscle metabolism (e.g., impaired glucose uptake and glycolysis), as well as muscle extrinsic, systemic acting factors including aging-related changes impairments in dietary food intake, absorption, and metabolism (Rosenberg et al., 1997). Physical training and a healthy lifestyle can slow down muscle loss during aging. However, this cannot prevent the loss of muscle mass and the associated reduction in quality of life in the long term. In addition, physical training in older age is further limited by increasing immobility, which elevates the risk of falls/injury. Other, effective therapies for aging-associated loss of muscle mass are currently not established (Lo, J, et al., 2020; Wu, P Y, et al. 2021).

Sarcopenia can also occur at younger age, for example in response to immobilization, loss of motor neuron function, immobilization (for example in casts, intensive care patients, astronauts) (Pietrocola et al., 2015) and can be linked to impairment and/or dysfunction in liver fat metabolism and associated hepatic diseases (such as non-alcoholic steatohepatitis, cirrhosis). For example cirrhosis is known to induce sarcopenia development and it is possible that impairments in hepatic fat metabolism also contribute to this process (Montano-Loza et al., 2014; Lee et al, 2016; Pasmans et al, 2021) Mechanistically, impairments in liver metabolism are known to induce catabolic metabolism in skeletal muscle, which in turn may enhance muscle wasting in such settings (Lee et al., 2016) Liver aging (not induced by liver disease) is also associated with impairments in hepatic fat metabolism (Gong et al., 2017; Ishizuka et al., 2020) NAFLD is the most prevalent liver disease in the world and its inflammatory sub-form, NASH, is nowadays the leading cause of cirrhosis and liver transplantation. The diseases are tightly linked to systemic, metabolic disease stages, such as diabetes mellitus and sarcopenia.

Four clinical disease states associated with impairment and/or dysfunction of liver fat metabolism and sarcopenia are particularly noteworthy:
(1) The loss of muscle mass in dietary treatment of patients with obesity (adipositas):

Obesity is a widespread disease in countries with high or increasing development status. It affects 650 million people worldwide in 2016, and according to the WHO, this number continues to rise sharply. Obesity leads to several diseases, such as cardiovascular disease, diabetes mellitus, and cancer. A guideline-based treatment for obesity is the implementation of weight-reducing diets, such as formula diets (Halle, et al. 2021). One of the main problems with this approach is the reduction in skeletal muscle maintenance on the diet. This cannot be adequately prevented even by concomitant physical training and additional protein intake, resulting in associated problems, such as further deterioration of the patients overall metabolic status, which contributes to a rapid relapse of obesity after discontinuation of the diet therapy (yo-yo effect).

Effective diet therapy to counteract the muscle loss of diet treatment for obesity is not currently available. Thus, the muscle atrophy associated with the dietary treatment of obesity (for example by formula diet supplemented with Vitamin-A) is a major limitation to the success of these therapies and cannot be sufficiently reduced by any measures to date.
(2) The loss of muscle mass in intensive care patients:

The loss of muscle preservation in intensive care patients develops due to artificial (parenteral) nutrition and immobilization. This muscle loss represents a very significant medical problem associated with increased mortality and prolonged convalescence in surviving patients (Kelmenson, et al. 2017). Intensive Care Unit-acquired weakness (ICU-AW) involves muscle atrophy acquired during intensive care treatment. It affects 30-67% of ICU patients. ICU-AW associates with poor prognosis of patients due to complications during weaning from ventilator machines and long-term increased mortality and morbidity in the post-inpatient course. In Germany, the annual number of ICU-treated patients increased by 37% from 2007 to 2015 to 736,444 in 2015 (Fleischmann-Struzek et al, 2019). There are currently no effective therapies for the prevention of muscle loss in intensive care patients. Clinical trials of nutritional approaches have not shown efficacy (Reinhold, et al. 2020).
(3) The loss of muscle mass and function during aging:

>10% of elderly people over the age of 65 are affected by sarcopenia. Sarcopenia associates with an increased risk of falls, and an increase in all-cause mortality. Aside from exercise, which is of limited use in the elderly, there are currently no efficient therapies to prevent aging associated sarcopenia.
(4) Genetic predisposition to impairments in fat metabolism and increases in triglyceride levels, such as mutant alleles of Ppar-a or other regulators of fat metabolism.

Current treatment options for impairments and/or dysfunction in liver fat metabolism and associated diseases, such as sarcopenia and liver diseases, such as NAFDL, are as follows:

The most consistent evidence for an effective sarcopenia treatment is derived from studies on physical exercise. Especially, resistance exercise seems to be effective in preventing age related muscle loss and increase muscle strength (Beckwee, et al., 2019; Peterson, et al., 2011; Peterson, et al., 2010; WHO. World Health Organisation—Global recommendations on physical activity for health 2010). However, exercise treatment is limited by immobilization and can thus not be applied to patients suffering from muscle wasting and immobilization.

Other experimental therapies for sarcopenia were investigated, including pharmacological interventions. Inhibition of anti-anabolic regulators like myostatin or activin are investigated as therapeutic targets. So far, too many side-effects and a lack of effectiveness have prevented the successful development of a therapy and some of the inhibitors have been discontinued in testing (Becker, et al., 2015; Woodhouse, et al. 2016, Rook, et al. 2017, Campbell, et al. 2017, Kwak, et al. 2019).

Further, hormone therapies agonizing androgen receptors and thereby stimulating muscle anabolism were shown to improve muscle mass (Sinha-Hikim, et al. 2008). However, safety concerns of testosterone treatment jeopardize the further development (e.g., increase of cancer risk) (Malafarina, et al., 2012). Testosterone further was shown to require a parenteral or intramuscular route of admiration, which is highly undesirable for drug development (Skinner, et al. 2018). Non-steroid agonists of androgen receptors were shown to be little or non-effective against sarcopenia (Dayal, et al. 2005; Dalton, et al., 2011; Papanicolau, et al. 2013). Ghrelin, a peptide hormone with anabolic effects on skeletal muscle and its analogues were shown to increase appetite and food intake associating with an increase of muscle mass (Nagaya, et al., 2004; Temel, et al., 2016). However, except of a cohort with chronic obstructive pulmonary disease (COPD) Ghrelin failed to improve muscle function (Nagaya, et al., 2005). In general, no conclusive consensus can be found whether pharmacological interventions are suitable or effective treatment options for sarcopenia.

Experimental data suggests positive effect of amino acid (AS) supplementation on muscle mass (Dickinson, et al., 2017; Walkerl, et al., 2011; Dillon, et al., 2009; Esmarck, et al. 2005). However, this has mainly been shown in combination with physical exercise and it is unclear whether AS supplementation alone is sufficient to improve muscle mass or function in aging (Godard, et al., 2002).

Vitamin D supplementation has received some attention as supplement for the treatment of sarcopenia (Rondanelli, et al., 2016; Verreijen, et al. 2014), although some studies also concluded no positive effect of vitamin D supplementation on muscle performance in old individuals (Latham, et al., 2003; Shea, et al., 2019; Levis, et al., 2016). The products used for sarcopenia treatment mainly include protein supplement, vitamin B12 supplement, vitamin D & calcium supplement.

Currently, physical exercise is the only available option for sarcopenia treatment. However, it has several limitations: (a) Especially with progressive sarcopenia, physical exercise becomes difficult to perform and increases the risk of injuries. (b) Physical exercise requires stringent participation in training sessions and a lack of motivation for physical exercise or adherence to training schedule reduces the effect.

The global market for sarcopenia treatments is expected to grow from around USD 2.76 billion in 2021 to nearly USD 3.8 billion in 2028 at a compound annual growth rate (CAGR) of 4.6%, according to analysts at Market Watch. China is attributed the largest market share at around 30%, followed by the U.S. at around 24.5% Market Watch. Sarcopenia Treatments Market Size 2022: Research Report by Global Growth Rate, Development Strategy, Recent Trends and Regional Demand till 2027 with Different Sectors and Countries Data). Mordor Intelligence even forecasts a CAGR of 5.71% (Mordor Intelligence SARCOPENIA TREATMENT MARKET—GROWTH, TRENDS, COVID-19 IMPACT, AND FORECASTS (2022-2027)) in a recent report. The reason for the market development is the increasing aging of the population and the growing interest in preventive healthcare, which ensures that the demand for nutritional supplements increases and the demand for sarcopenia treatment rises.

Efficient therapies that target impairments in hepatic fat metabolism, are not available (for review see Ferguson et al, 2019). Dietary treatments and lifestyle interventions may have a limited potential to slow progression of liver diseases such as NAFLD and NASH (El-Agroudy et al., 2019), but cannot effectively revert the route-cause of the diseases, e.g., impairments in liver fat metabolism. Pharmacologic therapies have focused on PPAR activation to improve liver fat metabolism, however, save and efficient PPAR activators remain still to be developed (Ferguson et al, 2021; Gross et al., 2017). Currently, glucagon-like peptide-1 receptor antagonists (GLP 1-RA) are considered one of the best candidates for pharmacological treatment of fatty liver diseases (Nevola et al., 2023), showing an induction of PPAR. However, GLP1-RA is an appetite suppressant which leads to an overall reduction in food intake by the patient and can thus significantly reduce the patient's quality of life. This often leads to poor compliance and often loss of weight, which is not necessarily desirable for the general state of health of the patient, e.g., of elderly and/or seriously ill patients.

Despite a wide variety of investigated approaches, effective prevention and/or treatment of an impairment and/or dysfunction in liver fat metabolism and associated medical conditions is currently still lacking. Thus, further developments are required for effective prevention and treatment of an impairment in liver fat metabolism and associated medical conditions, such as a loss in muscle mass and strength.

### SUMMARY OF THE INVENTION

In light of the prior art the technical problem underlying the present invention was to provide improved or alternative means for dietary treatments for the prevention and/or treatment of an impairment and/or dysfunction of liver fat metabolism or a medical condition associated therewith. Medical conditions associated with an impairment and/or dysfunction of liver fat metabolism include, for example:
(i) a muscle disease, such as loss of muscle mass and/or loss of strength in a subject, such as occurring during aging, in response to a diet, in response to immobilization, and/or in response to a liver disease associated with reduced liver fat metabolism, such as cirrhosis, nonalcoholic steatohepatitis (NASH) and nonalcoholic fatty liver disease (NAFLD),
(ii) a liver disease that leads to impairments and/or dysfunction in fat metabolism, such as cirrhosis, NASH and NAFLD,
(iii) a metabolic disease, such as metabolic syndrome, diabetes mellitus, insulin resistance and/or or elevated blood levels of triglycerides, LDL (low density lipoprotein, also termed LDL-cholesterin), cholesterin and/or total lipids that is induced and/or aggravated by an impairment and/or dysfunction in liver fat metabolism, and
(iv) an impairment and/or dysfunction of liver fat metabolism associated with a genetic predisposition to impairments in fat metabolism and/or elevated triglyceride levels in blood serum, such as sequence variants in the ppar-a gene or other genes that regulate liver fat metabolism.

Another problem underlying the invention was the provision of nutritional or dietary treatments for the prevention and/or treatment of an impairment and/or dysfunction of liver fat metabolism and/or a medical condition associated thereto such as loss in muscle mass and/or muscle strength in healthy subjects, and/or in patients, preferably without the application of any additional measures, such as physical exercise or pharmacological interventions, and preferably without inducing any harmful side effects.

Another problem underlying the present invention was the provision of nutritional or dietary treatments for the prevention and/or treatment of an aging associated impairment and/or dysfunction of liver fat metabolism and/or a medical condition associated thereto, such as a loss in muscle mass and/or muscle strength.

A further problem underlying the present invention was the provision of nutritional or dietary treatments for the prevention and/or treatment of an impairment and/or dysfunction of liver fat metabolism and/or a medical condition associated thereto, such as a loss in muscle mass and/or muscle strength or a liver disease such as NAFLD, NASH, cirrhosis that are the leading cause of liver failure.

Another problem underlying the present invention was the provision of improved or alternative means for dietary treatments that are not associated with a loss of weight, loss of appetite and/or low compliance.

In providing a solution to this problem, the invention seeks to avoid the disadvantages of the prior art.

The problems underlying the invention are solved by the features of the independent claims. Preferred embodiments of the present invention are provided by the dependent claims.

In one aspect the invention relates to a composition for use as a dietary treatment in preventing and/or treating an impairment and/or dysfunction of liver fat metabolism and/or a medical condition associated therewith, wherein the composition comprises dietary nutrients and is substantially free of vitamin A and/or derivatives thereof.

As used herein, the “patient” or “subject” may be a vertebrate. In the context of the present invention, the term “subject” includes both humans and animals, particularly mammals, and other organisms.

In embodiments the subject is a human subject. In embodiments the subject is an animal. In embodiments the animal is a farm animal such as a cow, a pig, a sheep, a goat, a horse, a chicken, an alpaca or a lama. In embodiments the animal is a pet animal such as a cat, a dog, a rabbit, a guinea pig, a rat, or a mouse or a bird such as a budgerigar or a parrot. In embodiments the animal is a cat.

The inventors surprisingly discovered that the Vitamin A-free dietary treatment (also termed herein “intervention” or “vitamin A free diet”; VAFD) of the present invention activates PPAR signaling, induces changes in the gut microbiome and improves absorption of further micronutrients in the gut, thereby activating and increasing liver fat metabolism and micronutrient absorption by the body. By mobilizing the storage form of Vitamin A (VA) in the liver, which is converted to retinoic acid (RA), RA targets are activated, such as PPAR-signaling as well as a reprogramming of the chromatin of hepatocytes that inhibits inflammation signaling pathways that are known to impair liver fat metabolism. Together, these RA effects contribute to improvements in liver fat metabolism as does the change in microbiome.

Thus, the VAFD of the present invention surprisingly has a strong capacity to prevent and/or treat impairment and/or dysfunction of liver fat metabolism and associated diseases such as muscle diseases, liver diseases and metabolic diseases without the need for application of any additional measures, such as physical exercise and/or pharmacological interventions, and without inducing any harmful side effects such as weakening of the immune system, hematopoiesis, eye damage (e.g., xerophthalmia, night blindness) or appetite and weight loss as observed for GLP-1 inhibitors. Further, the VAFD of the present invention surprisingly not only had a strong capacity to prevent and/or treat impairment and/or dysfunction of liver fat metabolism and associated diseases in human subjects but also animals such as farm animals or pet animals, e.g., cats often developing an impairment of liver fat metabolism and associated diseases such as a fatty liver over time.

In embodiments, the processed food product comprises an amount of dietary nutrients covering the daily requirements, or a part thereof, of a subject.

In one embodiment, the invention therefore relates to a composition for use as a dietary treatment in preventing and/or treating an impairment and/or dysfunction of liver fat metabolism and/or a medical condition associated therewith, wherein the composition comprises a processed food product comprising dietary nutrients and is substantially free of vitamin A and/or derivatives thereof.

In embodiments, the compositions of the present invention provide a complete and balanced diet for the subject, wherein solely the intake of Vitamin A or derivatives thereof is substantially reduced but the composition contains the essential nutrients required by the subject. By replacing the diet of a subject with the compositions of the present invention, a complete nutrition of the subject can be provided. Thus, advantageously, weight loss is avoided, which is undesirable in several patient groups, such as elderly or seriously ill patients. Further, in contrast to pharmacological interventions having an impact on liver fat metabolism, such as GLP-1 inhibitors, there is no suppression of appetite upon application of VAFD, advantageously resulting in a higher life quality and associated higher compliance of the patient. Further, the compositions of the present invention are suitable to replace commonly used animal feed which is typically enriched with vitamin A. Thereby, the compositions of the present invention advantageously provide a complete and balanced diet for an animal, wherein solely the intake of Vitamin A or derivatives thereof is substantially reduced. Furthermore, the compositions of the present invention comprise preferably processed food products that can be readily consumed by the subject, thereby directly replacing the usual food intake of the subject. This surprisingly simple application has the advantage of significantly increasing the patient's compliance, e.g., in comparison to a diet comprising meals that need to be prepared by the human subject or the owner of the animal or to pharmacological interventions with unwanted side effects.

To the knowledge of the inventors, a complete, nutritionally balanced and processed ready-to-use dietary composition essentially free of or without Vitamin A is not known in the prior art. The compositions of the present invention thus provide novel and inventive means for the prevention and/or treatment of impairment and/or dysfunction of liver fat metabolism and associated diseases.

In one embodiment, the invention relates to a composition for use as a dietary treatment in preventing and/or treating an impairment and/or dysfunction of liver fat metabolism and/or loss of muscle mass and/or a loss of muscle strength in a subject, wherein the composition comprises dietary nutrients and is substantially free of vitamin A and/or derivatives thereof.

In embodiments, the invention therefore relates to a composition for use as a dietary intervention in preventing and/or treating an impairment and/or dysfunction of liver fat metabolism and/or a medical condition associated thereto, wherein the composition comprises dietary nutrients necessary for regular nutrition of the subject and is substantially free of vitamin A and/or derivatives thereof.

In embodiments, the invention therefore relates to a composition for use as a dietary intervention in preventing and/or treating an impairment and/or dysfunction of liver fat metabolism, a loss of muscle mass and/or a loss of muscle strength in a subject, wherein the composition comprises dietary nutrients necessary for regular nutrition of the subject and is substantially free of vitamin A and/or derivatives thereof. In embodiments, the invention therefore to a composition for use as a dietary treatment in preventing and/or treating an impairment and/or dysfunction of liver fat metabolism, wherein the composition comprises one or more dietary nutrients and is substantially free of vitamin A and/or derivatives thereof.

In embodiments, the invention therefore to a composition for use as a dietary treatment in preventing and/or treating a loss of muscle mass and/or a loss of muscle strength in a subject, wherein the composition comprises one or more dietary nutrients and is substantially free of vitamin A and/or derivatives thereof.

In embodiments, the invention therefore relates to a composition for use as a dietary treatment in preventing and/or treating a loss of muscle mass and/or a loss of muscle strength in a subject, which is a consequence of an associated impairment and/or dysfunction of liver fat metabolism, wherein the composition comprises one or more dietary nutrients and is substantially free of vitamin A and/or derivatives thereof.

In embodiments, the invention is for use as a dietary treatment in preventing and/or treating a liver disease, a metabolic disease and/or sarcopenia.

In embodiments, the invention is for use as a dietary treatment in preventing and/or treating loss of muscle mass and/or a loss of muscle strength in a subject that are a consequence of an impairment and/or dysfunction of liver fat metabolism.

In one embodiment, the composition is a dietary composition. In one embodiment, the composition is a vitamin A-free nutritional composition. As used herein, the terms “dietary composition” or “nutritional composition” may be used interchangeably and relate to a composition comprising one or more dietary nutrients, as understood by a skilled person.

As used herein, dietary nutrients may be considered any substance used by an organism to survive, grow, and/or reproduce that is typically obtained by the subject through their diet. The requirement for dietary nutrient intake is fundamental to life and applies without limitation to animals, plants, fungi, and protists. As such, the compositions of the invention relate in embodiments, without limitation, to dietary products, food products, drinks, beverages, food supplements, or other dietary products suitable (for example) via ingestion by a subject, in which Vitamin A levels are substantially reduced, either to negligible levels, or in which vitamin A is absent. The term “dietary nutrients necessary for regular nutrition of the subject” refers to nutrients considered essential to survival, growth and/or reproduction of the subject, which are normally obtained through diet. The term dietary nutrients also includes medical nutrients, medical products and medical supplements that are used for the replacement of food intake (e.g., enteral or parenteral nutrition replacements) as well as formula diets used for treatment of medical conditions such as adipositas.

In a preferred embodiment, the term “substantially free of vitamin A and/or derivatives thereof” refers to the absence of vitamin A and/or derivatives thereof, or the presence of vitamin A and/or derivatives thereof at negligible levels, such as very low levels that do not hinder the technical effect of the present invention.

In one embodiment, the nutrients comprise carbohydrates, proteins, fats, vitamins, minerals, trace elements, secondary plant substances, derivatives and/or chemical precursors thereof and/or probiotic substances.

In one embodiment, the composition comprises an amount of vitamin A or derivatives thereof of equal or less than 5% of the daily requirement of Vitamin A or derivatives thereof of a subject, preferably equal or less than 2.5%, more preferably equal or less than 1%.

In one embodiment, the composition comprises an amount of vitamin A or derivatives thereof of 5, 4.5, 4, 3.5, 3, 2.5, 2,.5, 1, 0.5 or 0% of the daily requirement of Vitamin A or derivatives thereof of a subject.

In one embodiment, the composition comprises equal or less than 50 μg vitamin A or derivatives thereof, preferably equal or less than 25 μg, more preferably equal or less than 10 μg.

In one embodiment, the composition comprises an amount of 50, 45, 40, 35, 30, 25, 20, 15, 10, 5 or 0 μg vitamin A or derivatives thereof. In one embodiment, the dietary treatment comprises replacing the diet of the subject or parts thereof by the composition, preferably fully replacing the diet of the subject.

In one embodiment, the composition comprises a single meal portion. In one embodiment the composition comprises a single meal portion configured to replace the breakfast of a subject. In one embodiment the composition comprises a single meal portion configured to replace the lunch of a subject. In one embodiment the composition comprises a single meal portion configured to replace the dinner of a subject.

In one embodiment, the composition comprises two or more meal portions. In one embodiment, the composition comprises two or more meal portions configured to fully replace the diet of a subject for 8 to 24 hours, such as 8, 9, 10, 11, 12, 13, 14, 15, 16, 17, 18, 19, 20, 21, 22, 23, or 24 hours. In one embodiment, the composition comprises two or more meal portions configured to fully replace the diet of a subject for 16 to 24 hours.

In one embodiment, the composition comprises two or more meal portions configured to fully replace the diet of a subject for one day. In one embodiment, the composition comprises two or more meal portions configured to fully replace the diet of a subject for 1 to 7 days. In one embodiment, the composition comprises two or more meal portions configured to fully replace the diet of a subject for 1 to 14 days, such as 1, 2, 3, 4, 5, 6, 7, 8, 9, 10, 11, 12, 13 or 14 days. In one embodiment, the composition comprises two or more meal portions configured to fully replace the diet of a subject for 16 hours to 14 days, such as 16, 17, 18, 19, 20, 21, 22 or 23 hours or 1, 2, 3, 4, 5, 6, 7, 8, 9, 10, 11, 12, 13 or 14 days.

In one embodiment, the composition comprises two or more meal portions configured to fully replace the diet of a subject for 1 to 8 weeks. In one embodiment, the composition comprises two or more meal portions configured to fully replace the diet of a subject for 1 to 8 weeks, such as 1, 2, 3, 4, 5, 6, 7, or 8 weeks.

In one embodiment, the composition comprises two or more meal portions configured to fully replace the diet of a subject for 1 to 6 months. In one embodiment, the composition comprises two or more meal portions configured to fully replace the diet of a subject for 1 to 11 months, such as 1, 2, 3, 4, 5, 6, 7, 8, 9, 10 or 11 months. In one embodiment, the composition comprises two or more meal portions configured to fully replace the diet of a subject for 1 to 3 years. In one embodiment, the composition comprises two or more meal portions configured to fully replace the diet of a subject for 1 to 3 years, such as 1, 1.5, 2, 2.5 or 3 years.

In one embodiment, the composition is configured to fully replace the diet of a subject for 8 to 24 hours, wherein the composition applied on one day comprises an amount of vitamin A or derivatives thereof of equal or less than 5% of the daily requirement of Vitamin A or derivatives thereof of a subject, preferably equal or less than 2.5%, more preferably equal or less than 1%. In one embodiment, the composition is configured to fully replace the diet of a subject for 16 to 24 hours, wherein the composition applied on one day comprises an amount of vitamin A or derivatives thereof of equal or less than 5% of the daily requirement of Vitamin A or derivatives thereof of a subject, preferably equal or less than 2.5%, more preferably equal or less than 1%.

In one embodiment, the composition is configured to fully replace the diet of a subject for 1 to 7 days, wherein the composition applied on one day comprises an amount of vitamin A or derivatives thereof of equal or less than 5% of the daily requirement of Vitamin A or derivatives thereof of a subject, preferably equal or less than 2.5%, more preferably equal or less than 1%.

In one embodiment, the composition is configured to fully replace the diet of a subject for 1 to 14 days, wherein the composition applied on one day comprises an amount of vitamin A or derivatives thereof of equal or less than 5% of the daily requirement of Vitamin A or derivatives thereof of a subject, preferably equal or less than 2.5%, more preferably equal or less than 1%. In one embodiment, the composition is configured to fully replace the diet of a subject for 16 hours to 14 days, wherein the composition applied on one day comprises an amount of vitamin A or derivatives thereof of equal or less than 5% of the daily requirement of Vitamin A or derivatives thereof of a subject, preferably equal or less than 2.5%, more preferably equal or less than 1%.

In one embodiment, the composition is configured to fully replace the diet of a subject for 1 to 8 weeks, wherein the composition applied on one day comprises an amount of vitamin A or derivatives thereof of equal or less than 5% of the daily requirement of Vitamin A or derivatives thereof of a subject, preferably equal or less than 2.5%, more preferably equal or less than 1%.

In one embodiment, the composition is configured to fully replace the diet of a subject for 1 to 11 months, wherein the composition applied on one day comprises an amount of vitamin A or derivatives thereof of equal or less than 5% of the daily requirement of Vitamin A or derivatives thereof of a subject, preferably equal or less than 2.5%, more preferably equal or less than 1%.

In one embodiment, the composition is configured to fully replace the diet of a subject for 1 to 3 years, wherein the composition applied on one day comprises an amount of vitamin A or derivatives thereof of equal or less than 5% of the daily requirement of Vitamin A or derivatives thereof of a subject, preferably equal or less than 2.5%, more preferably equal or less than 1%. In one embodiment, the composition is configured to fully replace the diet of a subject for 8 to 24 hours, wherein the composition applied on one day comprises an amount of vitamin A or derivatives thereof of equal or less than 50 μg vitamin A or derivatives thereof, preferably equal or less than 25 μg, more preferably equal or less than 10 μg. In one embodiment, the composition is configured to fully replace the diet of a subject for 16 to 24 hours, wherein the composition applied on one day comprises an amount of vitamin A or derivatives thereof of equal or less than 50 μg vitamin A or derivatives thereof, preferably equal or less than 25 μg, more preferably equal or less than 10 μg

In one embodiment, the composition is configured to fully replace the diet of a subject for 1 to 7 days, wherein the composition applied on one day comprises an amount of vitamin A or derivatives thereof of equal or less than 50 μg vitamin A or derivatives thereof, preferably equal or less than 25 μg, more preferably equal or less than 10 μg.

In one embodiment, the composition is configured to fully replace the diet of a subject for 1 to 14 days, wherein the composition applied on one day comprises an amount of vitamin A or derivatives thereof of equal or less than 50 μg vitamin A or derivatives thereof, preferably equal or less than 25 μg, more preferably equal or less than 10 μg. In one embodiment, the composition is configured to fully replace the diet of a subject for 16 hours to 14 days, wherein the composition applied on one day comprises an amount of vitamin A or derivatives thereof of equal or less than 50 μg vitamin A or derivatives thereof, preferably equal or less than 25 μg, more preferably equal or less than 10 μg.

In one embodiment, the composition is configured to fully replace the diet of a subject for 1 to 8 weeks, wherein the composition applied on one day comprises an amount of vitamin A or derivatives thereof of equal or less than 50 μg vitamin A or derivatives thereof, preferably equal or less than 25 μg, more preferably equal or less than 10 μg.

In one embodiment, the composition is configured to fully replace the diet of a subject for 1 to 11 months, wherein the composition applied on one day comprises an amount of vitamin A or derivatives thereof of equal or less than 50 μg vitamin A or derivatives thereof, preferably equal or less than 25 μg, more preferably equal or less than 10 μg.

In one embodiment, the composition is configured to fully replace the diet of a subject for 1 to 3 years, wherein the composition applied on one day comprises an amount of vitamin A or derivatives thereof of equal or less than 50 μg vitamin A or derivatives thereof, preferably equal or less than 25 μg, more preferably equal or less than 10 μg.

Advantageously, by the compositions of the present invention, processed, ready-to-use compositions can be provided which can easily be prescribed to a subject for the required time period. As the compositions are preferably complete and nutritionally balanced, no harmful side effects, such as weight loss or reduction of the level of Retinol in the eye, occur, even after longer periods of application. Further, as the compositions are processed and ready-to-use, the long-term compliance of the subject to said diet can substantially be improved.

In one embodiment, upon replacement of the diet of the subject or parts thereof by the composition, no substantial reduction in retinol serum levels occurs in the subject.

In one embodiment, upon replacement of the diet of the subject or parts thereof by the composition, no substantial reduction in retinol serum levels and/or retinol levels in the eye occurs in the subject. In one embodiment, upon replacement of the diet of the subject or parts thereof by the composition, no substantial reduction in retinol levels in the eye occurs in the subject.

Surprisingly the compositions of the present invention, even when applied over longer periods of time, avoid harmful side effects associated with low Vitamin A levels in humans. In particular, the function of eyes and visual perception relies on constant levels of VA. Low levels of Vitamin A can lead to eye damage (e.g., xerophthalmia, night blindness). Surprisingly no reduction of Vitamin A levels in the eye is observed upon replacing a subjects' diet by the composition of the present invention, Thus, advantageously harmful side effects in particular associated with the eye can be avoided.

The invention relates in some embodiments to a medical treatment for treating and/or preventing the indicated medical condition, such as an impairment and/or dysfunction of liver fat metabolism and/or a medical condition associated thereto such as liver disease, loss of muscle mass and/or a loss of muscle strength.

In some embodiments, the method relates to a composition as described herein for use as a dietary treatment to modify liver fat metabolism, and/or glucose metabolism and insulin sensitivity of various organs and increase muscle mass and/or increase muscle strength in a subject.

In other embodiments, should the subject not receive any medical benefit from the method, the invention relates to a non-medical treatment to increase muscle mass and/or increase muscle strength in a subject by way of the inventive composition that comprises dietary nutrients and is substantially free of vitamin A and/or derivatives thereof. In other embodiments, should the subject not receive any medical benefit from the method, the invention relates to a non-medical treatment to increase muscle mass and/or increase muscle strength in a subject by way of the inventive composition that is a processed food product comprising an amount of nutrients covering the daily requirement of said nutrients or a part thereof of a subject and is substantially free of vitamin A and/or derivatives thereof. In non-medical embodiments the subject may not suffer or is not predicted to suffer from a loss of muscle mass and/or a loss of muscle strength. In non-medical embodiments the method may relate to a method of enhancing muscle mass and/or muscle strength.

The term vitamin A and derivatives (VA, CAS No. 68-26-8) covers all fatty compounds that have the activity of all-trans-retinol and its esters. The vitamin A family also includes about 50 carotenoids, of which about 12 are vitamin A active. Chemically, they are retinoids, which also include synthetic vitamin A analogues. Among others retinol (vitamin A1), retinal (vitamin A aldehyde), retinoic acids (vitamin A acids), retinyl esters (especially retinyl palmitate, retinyl propionate, retinyl stearate (vitamin A ester)), 3-dehydroretinol (vitamin A2) and provitamin A-carotenoids (α-carotene, β-carotene, β-cryptoxanthin) are summarized as vitamin A in humans.

Vitamin A is present in animal food products (especially liver, milk, eggs, fish) in the form of long-chain fatty acid esters of retinol (especially retinyl palmitate). Vegetable sources contain carotenoids (mainly carrots, spinach, kale). The vitamin A content in foods is uniformly expressed as retinol activity equivalent (RAE) or retinol equivalent (RE). 1 RAE is equivalent to 1 μg of all-trans-retinol, 2 μg of β-carotene (in oil), 12 μg of β-carotene in food, or 24 μg of other carotenoids acting as provitamin A. Where 1 RE corresponds to 1 μg of all-trans-retinol, 6 μg of β-carotene or 12 μg of other carotenoids acting as provitamin A. The recommended daily requirement of vitamin A for adults is between 0.8 and 1.0 mg RE (“Höchstmengenvorschläge für Vitamin A in Lebensmitteln und Nahrungsergänzungsmitteln”, Bundesinstitut für Risikobewertung (BfR), 24.09.2002). The actual daily requirement in humans depends on age, gender, and life circumstances. Women older than 19 years should consume an average of 0.8 mg RE, men up to 1 mg RE daily. Adolescent individuals have a slightly higher requirement. Young women 15 to 18 years of age should consume 0.9 mg RE per day and young women aged 13 to 14 years 1 mg RE daily. Young men aged 13 to 18 years should consume 1.1 mg. For pregnant women, a daily dose of 1.1 mg RE and for breastfeeding women 1.5 mg RE is recommended.

The body can hardly metabolize excess vitamin A, which is why it easily accumulates in the body, especially in the liver. Vitamin-A and its pro-vitamins are essential food components that can regulate muscle growth and regeneration as well as fat metabolism via retinoic acid (RA) signaling. Vitamin-A is known to influence muscle growth at young age, for example postnatal growth and marbling of skeletal muscle in young cattle (Harris et al., 2018). It has been reported that the induction of RA-signaling in muscle stromal cells (fibroadipogenic progenitors=FAP) can enhance skeletal muscle regeneration in young mice, but our invention shows that excessive amounts of VA impair muscle maintenance (FIG. 3) whereas VAFD in aged animals improves muscle maintenance and strength (FIG. 4).

Surprisingly, the Vitamin A-free dietary treatment (also termed herein “intervention” or “vitamin A free diet”; VAFD) of the present invention has a strong capacity to prevent the development of a loss of muscle strength and mass without the need for application of any additional measures, such as physical exercise and without inducing any harmful side effects, as shown within the examples. Side effects, preferably to be avoided, that can occur in humans when vitamin A levels are too low are a weakening of the immune system and hematopoiesis, which can lead to skin redness as well as typical eye damage (e.g., xerophthalmia, night blindness).

The VAFD of the present invention prevents the age-related activation of protein degradation in muscle, thereby better preserving muscle mass. Primarily, impaired lipid metabolism in the liver contributes to the increased muscle catabolismin old age, because of which protein catabolismin muscle increases to maintain the metabolic efficiency of the liver. VAFD breaks this catabolic cycle by epigenetically rejuvenating the aged liver, by activating PPAR signaling, and by inducing changes in the gut microbiome that activate liver fat metabolism. This associates with an improvement in liver lipid metabolism and a decrease in protein breakdown in muscle. Thereby, the VAFD does not reduce blood levels of vitamin A over the treatment period, preventing side effects. The intervention shows that VAFD-mediated changes in the gut microbiome in a way that induces liver fat metabolism. It also shows that mobilization of storage form from VA in liver, which are reverted to retinoic acid (RA) leads to activation of RA targets, such as PPAR-signaling as well as a reprogramming of the chromatin of hepatocytes that inhibits inflammation signaling pathways that are known to impair liver fat metabolism. Together, these RA effects contribute to improvements in liver fat metabolism as does the change in microbiome. VAFD is invented as a dietary way to achieve sustained activation (over weeks to months) of RA targets in liver and sustained changes in microbiome, which cannot be achieved by other diets such as starvation. In a preferred embodiment of the invention the composition can thus comprise probiotic substances for mediating changes in the gut microbiome involving but not limited to Proteobacteria of the Desulfovibrionaceae family (FIG. 2), which are known to induce impairments in fat metabolism and obesogenicity in response to high fat diet.

Probiotics are defined as live microorganisms that, when administered in sufficient quantity, (may) have health benefits for the host. Probiotic microorganisms mainly include representatives of lactobacilli, bifidobacteria and yeasts such as Saccharomyces boulardii. But also more specific strains of bacteria like butyrate producing Faecalibacterium prausnitzii or mucin degrading Akkermansia muciniphila are debated as probiotic treatment options. Importantly, these probiotics have shown to have substantially improve disease conditions of NAFLD and NASH (Brandi et al., 2017). They are used in the form of food supplements and drugs for the therapy and prevention of diseases. The mechanism of action may include competitive displacement of pathogenic agents by degradation of required substrates or occupation of receptors, production of antimicrobial substances, a decrease of the pH value, activation of the immune system in the host: Some probiotic microorganisms trigger signaling cascades in the context of adhesion to epithelial cells, which lead to the release of cytokines.

The VAFD of the present invention further preferably contains all or part of the necessary dietary nutrients for a balanced diet, but is substantially free of vitamin A. There is no evidence in the state of the art for an explicitly vitamin A-free diet to be applied in the context of the inventive treatment. On the contrary, the generally accepted doctrine states that an adequate supply of vitamin A is essential for health and that especially in the case of the elderly an adequate supply of vitamins must be ensured. A VAFD is currently not available, and it is not possible to naturally eat “vitamin A free”, as vitamin A occurs in a large variety of animal food products and vegetable precuts. Further, a VAFD can be applied at low cost as a ready-to-use product.

Vitamin-A free compositions can however be prepared as food products. Food products with no or low levels of vitamin A are, for example, without limitation, artichokes, asparagus, bamboo shoots, beans, most varieties, butter lettuce (light, green), cassava root (not flour), celery, celery root, cucumber (peeled), dill, ginger root, green beans, hearts of palm, iceburg lettuce, mushrooms (cooked, not shiitake), parsnips, potatoes (peeled), romaine lettuce, rosemary, water chestnuts, zucchini, (peeled), barley, hulled, buckwheat, corn, white or blue, hominy, white, masa, white, oats, rice, white flour if used in real, sourdough bread, apples, white-fleshed, bananas, medium ripe, blackberries, blueberries, cranberries, currants, dates, figs, black, grapes, lemons, limes, pomegranate, raisins, raspberries, strawberries, white peaches, proteins, beef, buffalo, chicken, game, wild, lamb, rabbit, turkey, white fish, agar seaweed, almonds, organic, apple cider vinegar, small amounts, arrowroot, avocado oil, refined, baking soda, barley, hulled, brazil nuts, buckwheat, butter (& ghee), cardamom, capers, carob, chestnuts, coconut aminos, coconut oil, refined, coconut sugar, coconut water, corn, white or blue, cumin, fenugreek, hazelnuts, honey, light colored, hummus, in moderation, maple syrup, nut/seed butter, oats, pecans, psyllium husk, rice, rice vinegar, walnuts, tapioca (and flour), tamarind, tea.

Vegetables with no or low vitamin A content include, without limitation, arrowhead, bamboo shoots, bamboo shoots, beans, pinto, burdock root, butterbur, cardoon, cauliflower raw, celeriac, chayote, corn, fungi, cloud ears, ginger root raw, gourd, hearts of palm, kanpyo, (dried gourd strips), lotus root, mountain yam, mushrooms, onion rings, breaded, parsnips, pepeao, potato flour, potatoes, radishes, salsify, seaweed, sesbania flower, tomatoes, turnips, waterchestnuts.

In the context of the present invention the term “processed” or “processed food product” relates to a composition comprising at least one, or preferably at least two or more, ingredients, such as the food products listed above, which have been processed and/or modified in some way by applying methods such as, without limitation, cutting, mixing, pressing, refining, grinding, crushing, cooking, freezing, baking, heating, smoking, curing, fermenting and/or drying, which are considered processing methods. In embodiments, processed food products may be ready-to-use products such as solid meals, liquid or semi-liquid meals, powders for reconstitution with a liquid, such as water, and/or ready-to-eat meals.

Compositions of the invention can be prepared by sourcing foods with no vitamin a and/or removing vitamin A from existing foods.

In one embodiment the composition is configured to be administered periodically, preferably at least once weekly, more preferably at least once daily, and most preferably two or more times daily.

In one embodiment the composition is configured to be administered orally, enterally, or parenterally to the subject.

In one embodiment the composition is configured to be administered orally, or parenterally to the subject.

In one embodiment, the medical condition associated with an impairment and/or dysfunction of liver fat metabolism is a liver disease such as liver cirrhosis, NASH and/or NALFD.

In one embodiment, the medical condition associated with an impairment and/or dysfunction of liver fat metabolism is a liver disease such as liver cirrhosis, NASH and/or NALFD.

In one embodiment, the medical condition associated with an impairment and/or dysfunction of liver fat metabolism is a muscle disease such as loss of muscle mass and/or strength and/or sarcopenia.

In one embodiment, the medical condition associated with an impairment and/or dysfunction of liver fat metabolism is a metabolic disease, such as metabolic syndrome, diabetes mellitus, insulin resistance and/or elevated blood levels of triglycerides, low density lipoprotein (LDL), cholesterin and/or total lipids.

In one embodiment, the impairment and/or dysfunction in liver fat metabolism occurs in a subject ≥30 years, more preferably in a subject ≥40 years and most preferably in a subject ≥60 years. In one embodiment the impairment and/or dysfunction in liver fat metabolism, the loss of muscle mass and/or loss of muscle strength in the subject occurs in a subject ≥30 years, more preferably in a subject ≥40 years and most preferably in a subject ≥60 years.

In one embodiment, the impairment and/or dysfunction in liver fat metabolism occurs in an elderly subject, such as ≥60 years, ≥65 years, ≥70 years, ≥75 years, ≥80 years, or ≥85 years. In one embodiment the impairment and/or dysfunction in liver fat metabolism, the loss of muscle mass and/or loss of muscle strength occurs in an elderly subject, such as ≥60 years, ≥65 years, ≥70 years, ≥75 years, ≥80 years, or ≥85 years.

In one embodiment, the impairment and/or dysfunction in liver fat metabolism is associated with reduced mobility of the subject. In one embodiment the impairment and/or dysfunction in liver fat metabolism, the loss of muscle mass and/or loss of muscle strength in the subject is associated with reduced mobility of the subject.

In one embodiment, the impairment and/or dysfunction in liver fat metabolism is associated with an intensive care intervention, intensive care unit-acquired weakness (ICU-AW), hospitalization, prolonged period of reduced mobility, prolonged bed-ridden condition, perioperative loss of muscle mass and/or muscle strength, immobilization of injured body parts and/or loss of motor neuron function.

In one embodiment, the impairment and/or dysfunction in liver fat metabolism, the loss of muscle mass and/or loss of muscle strength in the subject is associated with an intensive care intervention, intensive care unit-acquired weakness (ICU-AW), hospitalization, prolonged period of reduced mobility, prolonged bed-ridden condition, perioperative loss of muscle mass and/or muscle strength, immobilization of injured body parts and/or loss of motor neuron function.

In one embodiment, the impairment and/or dysfunction in liver fat metabolism is associated with a reduced and/or altered food intake and/or malnutrition. In one embodiment the impairment and/or dysfunction in liver fat metabolism, the loss of muscle mass and/or loss of muscle strength in the subject is associated with a reduced and/or altered food intake and/or malnutrition.

In one preferred embodiment the reduced and/or altered food intake is related to a dietary intervention.

In one preferred embodiment the dietary treatment is related to an obesity-related metabolic disease.

In one preferred embodiment the dietary treatment is in the form of a formula diet and is related to an obesity related disease.

In one embodiment, the impairment and/or dysfunction of liver fat metabolism is associated with cachexia caused by cancer, age of the subject, a genetic predisposition of the subject to impairments in liver fat metabolism, rheumatoid arthritis, chronic renal insufficiency, chronic heart failure, acquired immune deficiency syndrome (AIDS), chronic lung diseases with respiratory insufficiency, chronic obstructive pulmonary disease (COPD) and/or diabetes mellitus type I.

In one embodiment the loss of muscle mass and/or loss of muscle strength in the subject is associated with cachexia caused by cancer, high age of the subject, rheumatoid arthritis, chronic renal insufficiency, chronic heart failure, acquired immune deficiency syndrome (AIDS), chronic lung diseases with respiratory insufficiency, chronic obstructive pulmonary disease (COPD) and/or diabetes mellitus type I.

In one embodiment the loss of muscle mass and/or loss of muscle strength in the subject is associated with an impairment and/or dysfunction of the liver metabolism.

In one preferred embodiment the impairment and/or dysfunction of the liver metabolism is associated with a liver disease, such as NASH, NAFLD, liver cirrhosis and/or liver aging.

In one preferred embodiment the impairment and/or dysfunction of liver fat metabolism, loss of muscle mass and/or loss of muscle strength in the subject is associated with metabolic diseases such as metabolic syndrome, diabetes mellitus, insulin resistance.

In addition to sarcopenia in old age, the indications obesity and the so-called intensive care unit-acquired weakness (ICU-AW) are particularly suitable for VAFD. The incidence of sarcopenia in the elderly is 10% in >60-year-olds. Estimates are that by 2050, one in six people in the world or one in four people in Europe and North America will be 65 years of age or older. Currently, the number is estimated to be about one billion worldwide, doubling by 2050. VAFD could be used, for example, in elective hip replacement surgery in elderly patients to prevent perioperative loss of muscle mass and improve postoperative mobilization. Obesity affected 650 million people worldwide in 2016, and this number continues to rise sharply unabated, according to the WHO. According to guidelines, therapy currently involves high-protein diets combined with physical training. However, the muscle atrophy associated with obesity is a major limitation of treatment success and has not been adequately reduced by interventions to date. Intensive Care Unit-acquired weakness (ICU-AW) affects 30-67% of ICU patients and is associated with poor patient prognosis. In such embodiments the invention compromises medical diets (enteral and/or parenteral medical diets) as well as formula diets.

Increases in intake or metabolism of VA have also been reported to elicit negative effects on liver metabolism, such as impairments in hepatic lipid metabolism, promoting the development of liver disease, liver failure, cirrhosis, and systemic metabolic diseases such as decreases in insulin sensitivity and glucose metabolism, metabolic syndrome, diabetes mellitus. Both cirrhosis and impairments in hepatic fat metabolism can lead to sarcopenia development. Mechanistically, impairments in liver metabolism are known to induce catabolic metabolism in skeletal muscle, which in turn can enhance muscle wasting in such settings. Liver aging (not induced by liver disease) is also associated with impairments in hepatic fat metabolism. Such conditions and diseases related to an impairment and/or dysfunction of the liver metabolism thus represent further indications for a dietary intervention by the VAFD. Surprisingly the inventors showed that VAFD has a strong potential to reactivate liver fat metabolism by (i) activating PPAR signaling in liver, (ii) reprogramming the chromatin structure of hepatocytes in that way that pro-inflammatory pathways that impair liver fat metabolism are suppressed, and (iii) by enhancing bacterial species in gut microbiome that enhance liver fat metabolism (FIG. 2). The combination of these different pathways of liver fat metabolism improvement leads to strong reduction in the accumulation of fatty acids in liver of aged mice thereby preventing induction of sarcopenia.

A further indication associated with a loss of muscle mass and/or muscle strength is cachexia. The term cachexia is used to describe pathological weight loss. In cachexia, there is not only a complete depletion of the storage fat depots, but also a generalized atrophy with a gradual loss of function of the organs. Important structural components such as the building fat and the musculature are also degraded. Causes of cachexia include but are not limited to consuming chronic diseases such as cancer and acquired immune deficiency syndrome (AIDS), high age of the subject, rheumatoid arthritis, chronic renal insufficiency, chronic heart failure, chronic lung diseases with respiratory insufficiency, chronic obstructive pulmonary disease (COPD)diabetes mellitus type I. Cachexia is considered one of the factors contributing to premature death of the patients.

These indications represent preferred embodiments of the use of the invention as a dietary treatment or intervention in preventing and/or treating a loss of muscle mass and/or a loss of muscle strength in a subject.

In one aspect, the invention relates to a method for reducing the vitamin A intake of a subject without substantially reducing Retinol serum levels and/or Retinol levels in the eye of the subject, comprising replacing the diet of the subject or parts thereof by a composition, wherein the composition is a processed food product comprising an amount of nutrients covering the daily requirement of said nutrients or a part thereof of a subject, and that is substantially free of vitamin A and/or derivatives thereof

In one aspect the invention relates to method for reducing the vitamin A intake of a subject without substantially reducing Retinol serum levels of the subject, comprising replacing the diet of the subject or parts thereof by a composition comprising nutrients necessary for regular nutrition of the subject and that is substantially free of vitamin A and/or derivatives thereof.

The features of the invention relating to the composition for use as a dietary intervention as described herein, are relevant to and considered disclosed in combination with other aspects of the invention, such as the method for reducing the vitamin A intake of a subject without reducing Retinol serum levels of the subject, and vice versa. Features defining methods of medical treatment may be used to define non-medical methods, if applicable in either context, and vice versa. For example, the composition fur use as a dietary intervention are immediately relevant to the other aspects of the invention and may be used to characterize them appropriately, as understood by a skilled person.

### FIGURES

FIG. 1: Induction of VitA metabolizing enzymes in freshly isolated fibro-adipogenic progenitors (FAPs) from aged vs. young mice.

FIG. 2: Vitamin A free diet (VAFD) reduces the cecal abundancies of proteobacteria belonging to the family of Desulfovibrionaceae.

FIG. 3: Vitamin A (VA) overfeeding impairs skeletal muscle maintenance.

FIG. 4: Dietary depletion of vitamin-A (VA) ameliorates aging-associated impairments in skeletal muscle function.

FIG. 5: Supplementary data to dietary depletion of depletion of vitamin-A (VA) ameliorating aging-associated impairments in skeletal muscle function.

FIG. 6: Dietary depletion of vitamin-A (VAFD) does not change retinoic acid (RA) signaling in skeletal muscle and VA levels in Eyes.

FIG. 7: VAFD mobilizes hepatic VA storages and rescues aging-associated impairments in liver fat metabolism.

FIG. 8: Supplementary data to VAFD mobilizing hepatic VA storages and rescues aging-associated impairments in liver fat metabolism.

FIG. 9: VAFD-mediated rescue in fat metabolism of aged liver involve Pparα/Fgf21—activation and chromatin silencing of fat metabolism suppressive pathways.

FIG. 10: VAFD ameliorates metabolic compromise, protein catabolismand maintenance of structural proteins of muscle contraction in aging muscle.

### DETAILED DESCRIPTION OF THE FIGURES

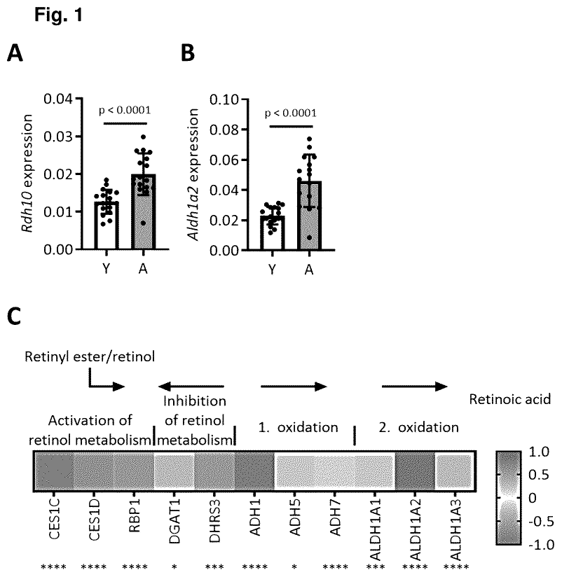

FIG. 1: A, Relative quantification of mRNA expression of the Retinol Dehydrogenase 10 (Rdh10) and the Aldehyde Dehydrogenase 1 Family Member A2 (Aldh1a2) in FAPs of young (3-5 months) and aged (22-25 months) mice. Expression values were calculated relative to Actin Beta (Actb). B, Heatmap depiction of Log2 fold changes of the indicated proteins of the VitA metabolic pathway as determined by proteome analysis of freshly isolated FAPs from aged (22 months) vs. young (3 months) mice (n=6 per group). Arrows indicate the direction of the enzymatic activity in terms of retinoic acid (RA) synthesis. Asterisks indicate q-value with *q<0.05, ***q<0.001, ****q<0.0001.

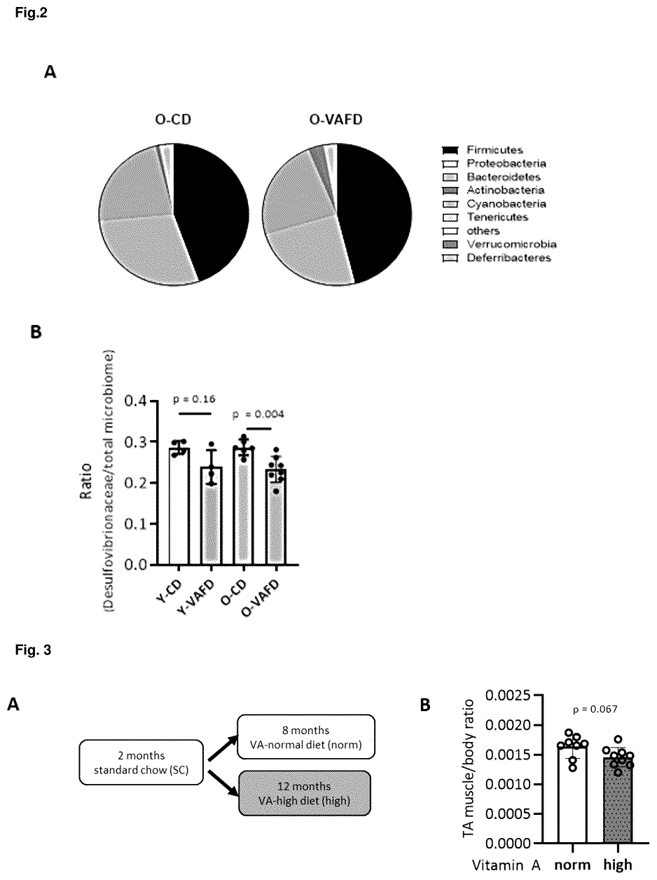

FIG. 2: A, Microbiome composition in ceacum of aged (27 months) mice after receiving a vitamin A control diet (CD, 25 000 IU/kg) or vitamin A free diet (VAFD, <120 IU/kg) for 8 months (n=6 for O-CD, n=8 for O-VAFD). B, Ratio of proteobacteria belonging to the family of Desulfovibrionaceae in ceacum of young (10 months) and aged (27 months) mice after receiving a vitamin A control diet (CD, 25 000 IU/kg) or vitamin A free diet (VAFD, <120 IU/kg) for 8 months (n=4 for Y-CD, n=4 for Y-VAFD, n=6 for O-CD, n=8 for O-VAFD). Statistics: For A mean values are shown. For B data is represented as mean±SD. Normal distribution of data was tested with the Shapiro-Wilk test. Statistical significance was calculated using one-way ANOVA with corrections for multiple testing.

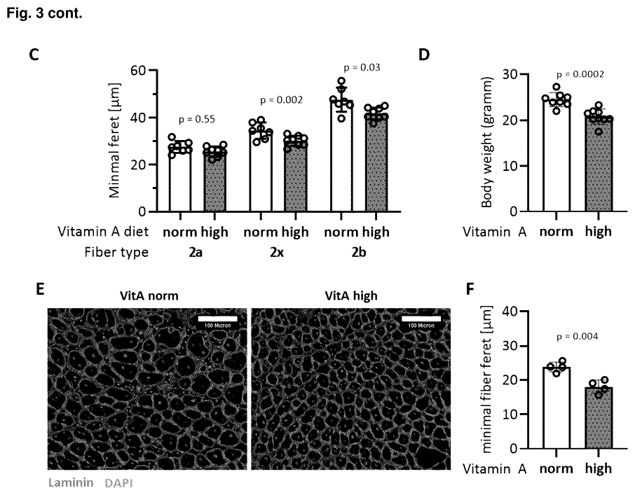

FIG. 3: A, Schematic outline of experimental groups and feeding period. Young mice (2 months) received a vitamin A normal diet (norm, 25 000 IU/kg) or vitamin A high diet (high, 333 000 IU/kg) for 12 months. B, TA-muscle weight to body weight ratio of adult mice on indicated vitamin A diets (n=8 for norm, n=9 for high). C, Quantification of fiber type sizes (minimal ferret) in TA-muscles of aged mice on indicated vitamin A diets (n=7 for norm, n=8 for high). D, Whole body weight of adult mice that received a vitamin A normal diet (norm, 25 000 IU/kg) or vitamin A high diet (high, 333 000 IU/kg) for 12 months. (n=8 for norm, n=9 for high). E, Representative image of immunostaining on TA-muscle cross sections visualizing laminin (red) in regenerating areas 10 days post injury by intramuscular cardiotoxin injection. Nuclei are counterstained with DAPI (blue). Scale bar: 100 μm. F, Quantification of fiber size in regenerating areas described in (E). Statistics: Data is represented as mean±SD. Normal distribution of data was tested with the Shapiro-Wilk test. Statistical significance was calculated using Welchs's t-test (B, D, F) or two-way ANOVA (C) with corrections for multiple testing.

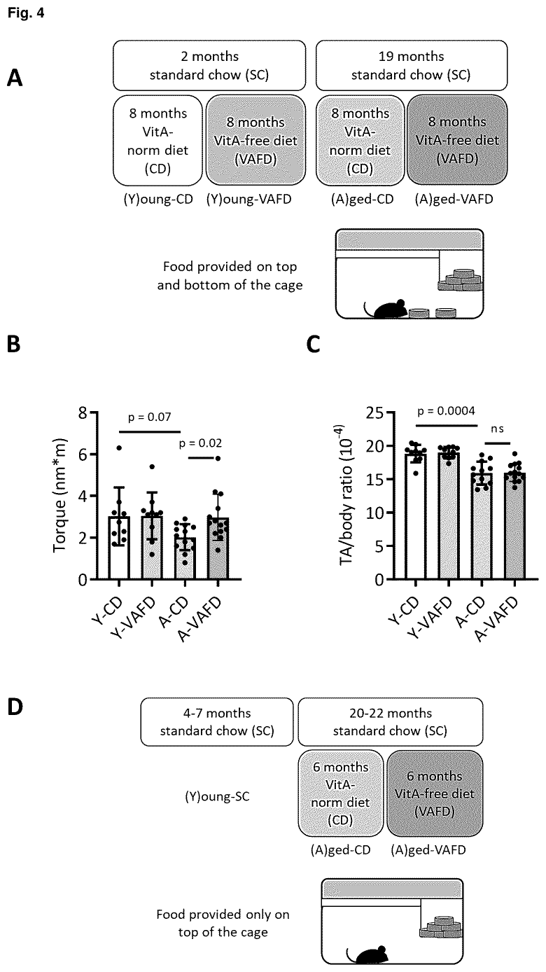

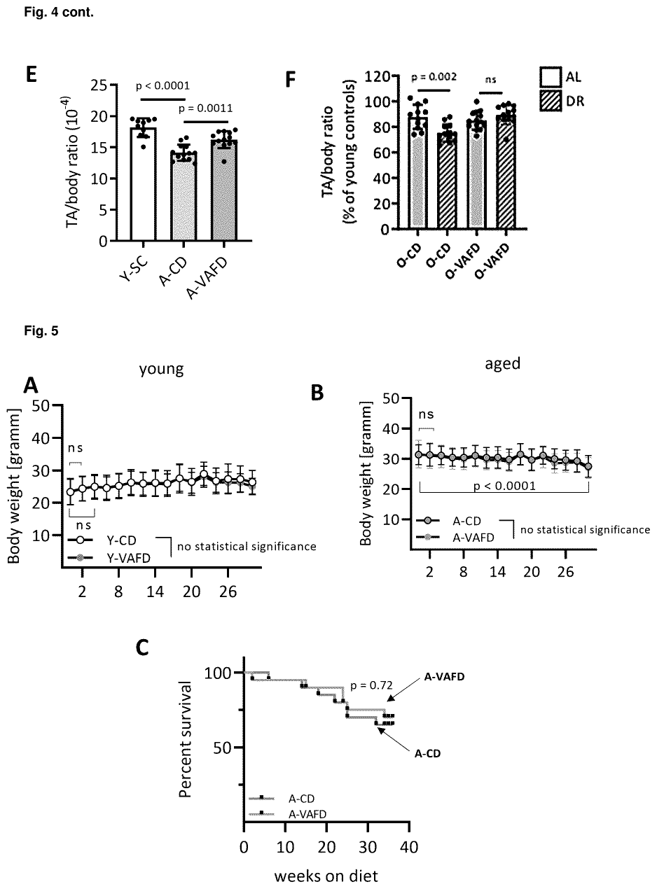

FIG. 4: A, Schematic outline of experimental groups and feeding period of hardened food with facilitated access to food. Young (2 months) and aged mice (19 months) received a vitamin A normal diet (CD, 25 000 IU/kg) or vitamin A free diet (VAFD, <120 IU/kg) for 8 months. B & C Plantar flexion tetanic torque (B) and TA-muscle weight to body weight ratio (C) of young (10 months) and aged (27 months) mice after receiving a vitamin A control diet (CD, 25 000 IU/kg) or vitamin A free diet (VAFD, <120 IU/kg) for 8 months with facilitated access to food as described in A (B: n=9 for adult-CD, n=10 for adult-VAFD, n=12 for A-CD, n=14 for A-VAFD, C: n=10 for adult-CD, n=10 for adult-VAFD, n=12 for A-CD, n=14 for A-VAFD). D, Schematic outline of experimental groups and feeding period of hardened food without facilitated access to food (mimicking of mild calorie restriction). Aged mice (20-22 months) received a vitamin A control diet (CD, 25 000 IU/kg) or vitamin A free diet (VAFD, <120 IU/kg) for 6 months. Young mice (4-7 months) were maintained on standard chow (SC) with identical Vitamin content and nutritional composition as the CD. E, TA-muscle weight to body weight ratio of aged (26-28 months) mice after receiving a vitamin A control diet (CD, 25 000 IU/kg) or vitamin A free diet (VAFD, <120IU/kg) for 6 months without facilitated access to food as described in D or young mice (4-7 months) on standard chow (n=10 for young, n=13 for aged). F, Comparison of TA to body weight rations of aged mice on indicated diet regimes as % of young control mice. Statistics: Data is represented as mean±SD. Normal distribution of data was tested with the Shapiro-Wilk test. Statistical significance was calculated using one-way ANOVA with corrections for multiple testing.

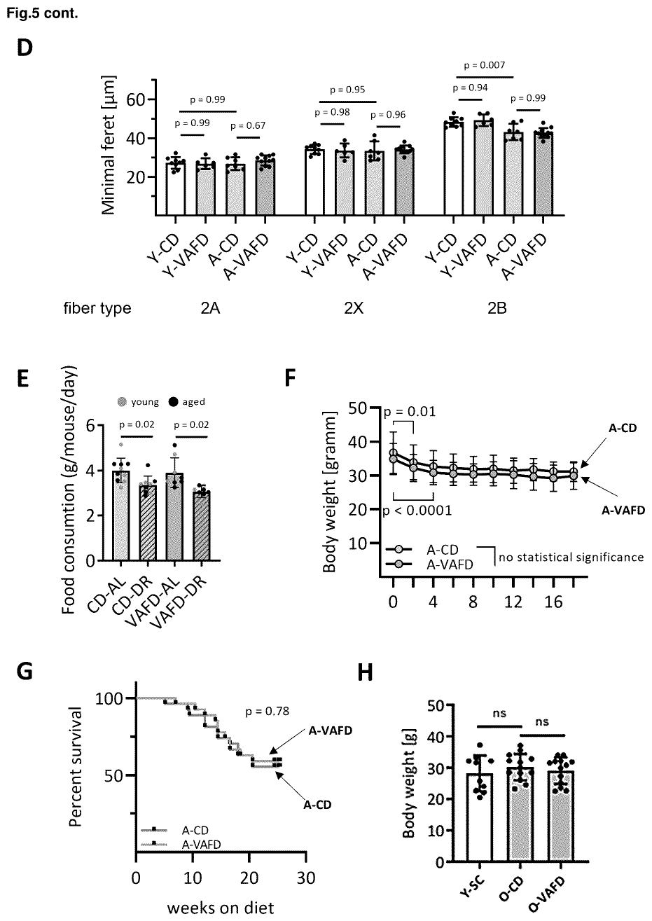

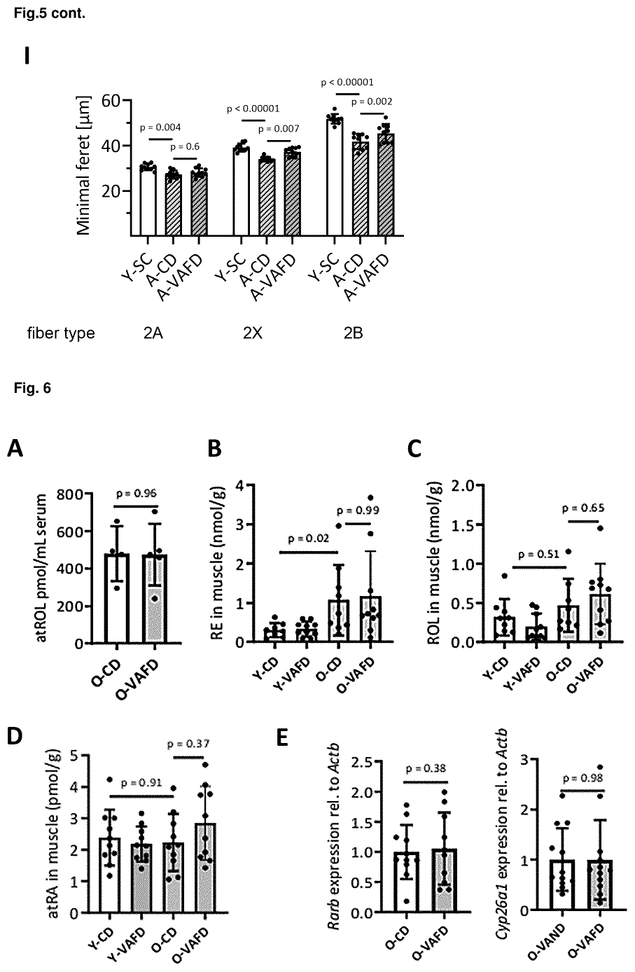

FIG. 5: A, Body weights of young ((A), 2 months) and aged ((B), 19 months) mice after receiving a vitamin A control diet (CD, 25 000 IU/kg) or vitamin A free diet (VAFD, <120 IU/kg) for 8 months with facilitated access to food as described in FIG. 3A. C, Survival of aged mice on indicated vitamin A diets (start of diet at the age of 19 months) during a period of 8 months with facilitated access to food as described in FIG. 3A. (n=20). D, Quantification of myofiber type sizes (minimal ferret) in TA-muscles of young (10 months) and aged (27 months) mice after receiving a vitamin A control diet (CD, 25 000 IU/kg) or vitamin A free diet (VAFD, <120 IU/kg) for 8 months with facilitated access to food as described in FIG. 1A (n=10 for Y-CD, n=7 for Y-VAFD, n=8 for A-CD, n=11 for A-VAFD). E, Food consumption/day of young (6-7 months) and aged (22-25 months) mice that were provided with facilitated access to food (AL) as described in FIG. 1A or only via the pellet rack on top of the cage (DR) as described in FIG. 1D. F, Body weights of aged mice (20-22 months) after receiving a vitamin A normal diet (CD, 25 000 IU/kg) or vitamin A free diet (VAFD, <120 IU/kg) during a period of 4 months without facilitated access to food as described in FIG. 3D. G, Survival of aged mice on indicated vitamin A diets (start of diet at the age of 20-22 months) during a period of 6 months without facilitated access to food as described in FIG. 3D. (n=27). H, Body weights of aged mice (26-28 months) that received a vitamin A normal diet (CD, 25 000 IU/kg) or vitamin A free diet (VAFD, <120 IU/kg) for 6 months without facilitated access to food as described in FIG. 3D and young mice (4-7 months) that were maintained on a standard chow. I, Quantification of myofiber type sizes (minimal ferret) in TA-muscles of aged mice on indicated vitamin A diets without facilitated access to food as described in FIG. 3D and young mice (4-7 months) that were maintained on a standard chow. (n=10 for all groups). Statistics: Data is represented as mean±SD. Normal distribution of data was tested with the Shapiro-Wilk test. Statistical significance was calculated using two-way ANOVA with corrections for multiple testing (A, B, D, F, I), one way ANOVA with corrections for multiple testing (E, H) or Mantel-Cox test (C, G).

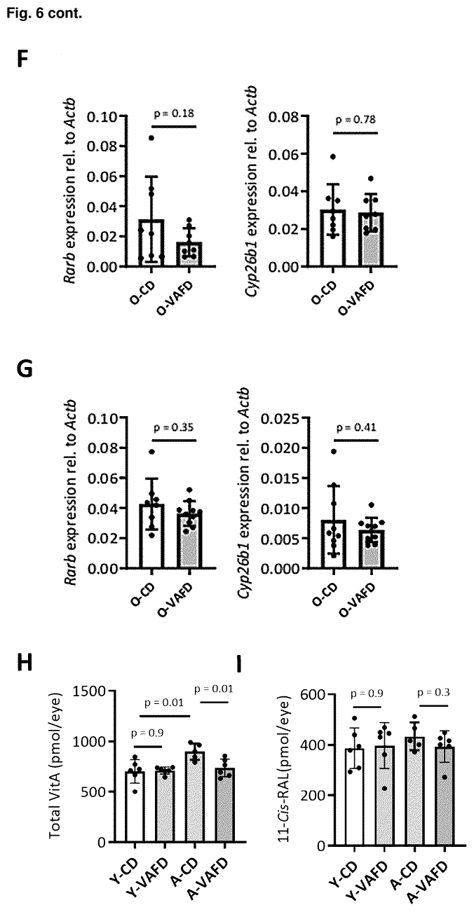

FIG. 6: A, Quantification of all-trans retinol (atROL) in blood serum of aged mice (26-28 months) after receiving a vitamin A control diet (CD, 25 000 IU/kg) or vitamin A free diet (VAFD, <120 IU/kg) for 6 months without facilitated access to food as described in FIG. 3D (n=4 for O-CD, n=5 for O-VAFD). B-D, Quantification of retinyl esters (RE), B), all-trans retinol (atROL), A) and all-trans retinoic acid (atRA), D) in gastrocnemius muscles of young (10 months) and aged (27 months) mice after receiving a vitamin A control diet (CD, 25 000 IU/kg) or vitamin A free diet (VAFD, <120 IU/kg) for 8 months with facilitated access to food as described in FIG. 3A (n=8-10 for all groups). E-G, Quantification of mRNA expression of retinoic acid target genes Rarb and Cyp26a1 in freshly isolated muscle stem cells (E), fibro-adipogenic progenitors (D) and full tibialis anterior muscle biopsies (G) of aged mice (26-28 months) after receiving a vitamin A control diet (CD, 25 000 IU/kg) or vitamin A free diet (VAFD, <120 IU/kg) for 6 months without facilitated access to food as described in FIG. 3D. Expression was calculated relative to Actb. H, I, Quantification of total vitamin A (H) and 11-cis retinal (I) in whole eyes of young and aged mice after receiving a vitamin A control diet (CD, 25 000 IU/kg) or vitamin A free diet (VAFD, <120 IU/kg) for 8 months (n=6 for all groups). Statistics: Data is represented as mean±SD. Normal distribution of data was tested with the Shapiro-Wilk test. Statistical significance was calculated using Welchs's t-test (A, E, F, G) or one way ANOVA with corrections for multiple testing (B, C, D, H, I).

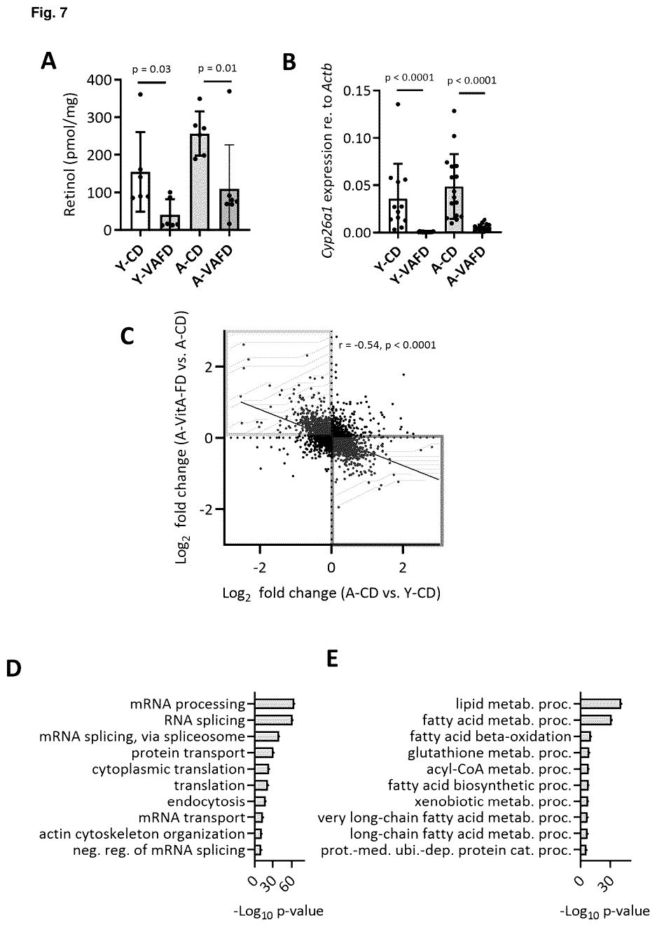

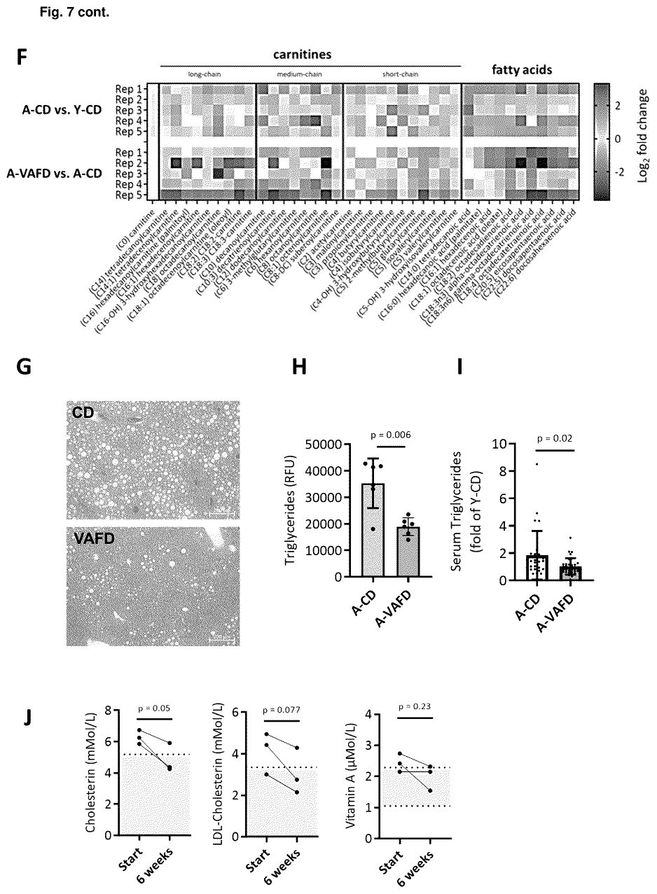

FIG. 7: A, Quantification of retinol in the liver of young and aged mice after receiving a vitamin A control diet (CD, 25 000 IU/kg) or vitamin A free diet (VAFD, <120 IU/kg) for 8 months with facilitated access to food as described in FIG. 4A (n=6 for all groups). B, Quantification of mRNA expression of retinoic acid target gene Cyp26a1 in full liver biopsies of young and aged mice after receiving a vitamin A control diet (CD, 25 000 IU/kg) or vitamin A free diet (VAFD, <120 IU/kg) for 8 months. Expression was calculated relative to Actb. C, x-y plot of log 2 fold changes of protein abundancies determined by mass spectrometry in the liver of young and aged mice after receiving a vitamin A control diet (CD, 25 000 IU/kg) or vitamin A free diet (VAFD, <120 IU/kg) for 8 months with facilitated access to food as described in FIG. 4A. Comparison shows A-CD vs. Y-CD (x-axis) and A-VAFD vs. A-CD (y-axis). Solid line indicates linear regression (n=5 for all groups). D, Gene ontology analysis (biological processes) of proteins that are significantly (q<0.05) up-regulated in the liver during aging but significantly down-regulated in livers of aged mice that received a VAFD compared to a CD diet (blue square in C). E, Gene ontology analysis (biological processes) of proteins that are significantly (q<0.05) down-regulated in the liver during aging but significantly up-regulated in livers of aged mice that received a VAFD compared to a CD diet (purple square in C). F, Quantification of fatty acids in the liver of young and aged mice on indicated vitamin A diets (same samples as in A, n=5 for all groups). G, H, I, Representative H&E images of liver cross-sections and triglyceride measurements in (H) full liver biopsies (n=6 for all groups) and (I) blood plasma (n=21 for Y-CD, n=21 for Y-VAFD, n=27 for A-CD and n=33 for A-VAFD) of aged mice after receiving a vitamin A control diet (CD, 25 000 IU/kg) or vitamin A free diet (VAFD, <120 IU/kg) for 8 months. J, Quantification of blood plasma levels of total Cholesterin, LDL-Cholesterin (also termed low density lipoprotein, LDL) and Vitamin A in 3 human volunteers, who maintained a substantially vitamin A free diet for 6 weeks. Gray area represents the desired physiological range for healthy adults. Statistics: Data is represented as mean±SD. Normal distribution of data was tested with the Shapiro-Wilk test. Statistical significance was calculated using Welchs's t-test (A, B, H, I), Paired t-test (J) or Fisher's Exact test with corrections for multiple testing (D, E).

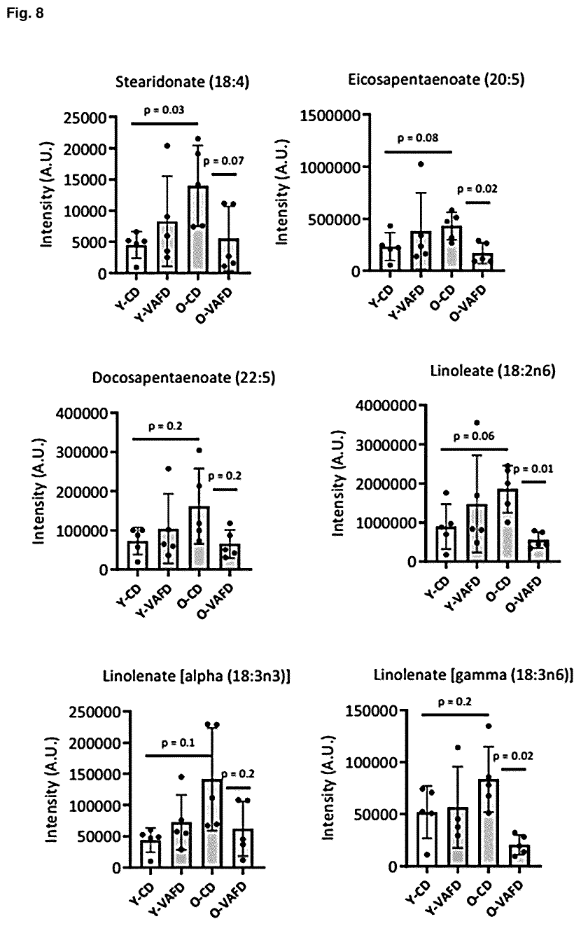

FIG. 8: Quantification of polyunsaturated fatty acids in the liver of young (10 months) and aged (27 months) mice after receiving a vitamin A control diet (CD, 25 000 IU/kg) or vitamin A free diet (VAFD, <120 IU/kg) for 8 months with facilitated access to food as described in FIG. 2A Statistics: Data is represented as mean±SD. Normal distribution of data was tested with the Shapiro-Wilk test. Statistical significance was calculated using one-way ANOVA or Kruskal-Wilis test with corrections for multiple testing.

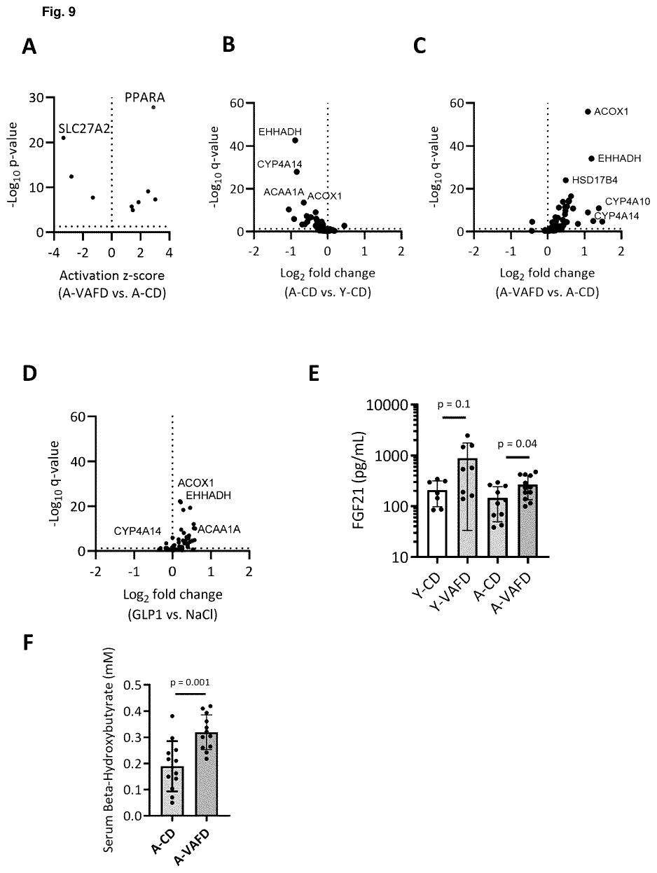

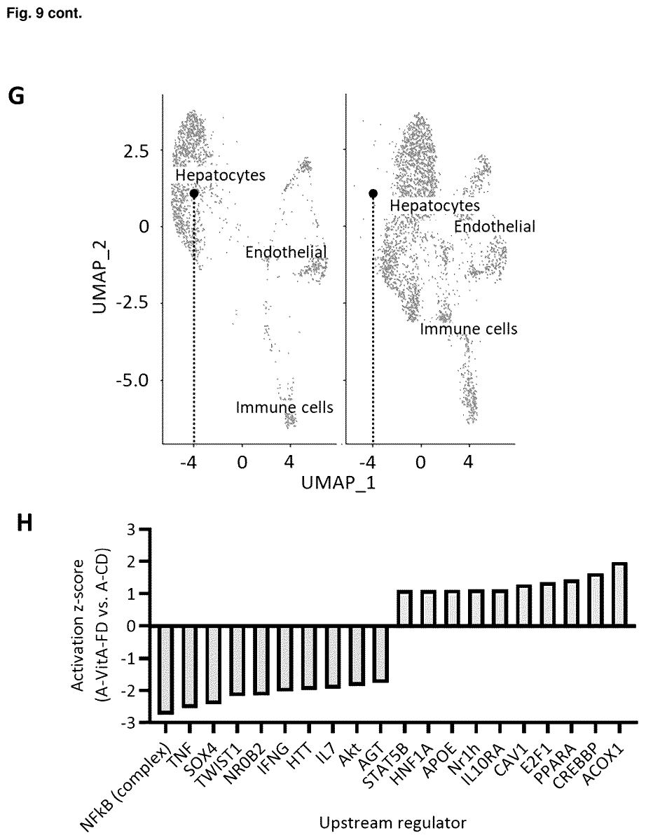

FIG. 9: A, Upstream regulator analysis (Qiagen IPA) of differentially abundant proteins in the liver of aged mice that were maintained for 8 months on a vitamin A free diet (VAFD, <120 IU/kg) vs. a control diet (CD, 25 000 IU/kg) (n=5 for all groups). B-D, Log2 fold changes of proteins encoded by PPAR-alpha target genes in the liver comparing (B) A-CD vs. Y-CD, (C) A-VAFD vs. A-CD and (D) GLP1-RA vs NaCl. E, F, Quantification of FGF21 (E) and beta-hydroxybutyrate (F) levels in blood plasma of aged mice after receiving a vitamin A control diet (CD, 25 000 IU/kg) or vitamin A free diet (VAFD, <120 IU/kg) for 8 months (E: n=10 for A-CD, n=12 for A-VAFD, F: n=12 for A-CD and A-VAFD). G, Cluster map (UMAP) of 3 most abundant cell type populations identified by single nuclei ATACseq in livers of aged (27 months) mice fed with a CD or a VAFD for 8 months. Dotted black line is an orientation help marking the same x-y position in G. H, Upstream regulator analysis (Qiagen IPA) of differentially accessible chromatin regions determined by single nuclei ATACseq in E. Top 10 ranked regulators with a significantly positive or negative activation z-score are shown. Statistics: For (E, F) data is represented as mean±SD. Normal distribution of data was tested with the Shapiro-Wilk test. Statistical significance was calculated using Fisher's Exact test with corrections for multiple testing (A-D) and Welchs's t-test (E, F).

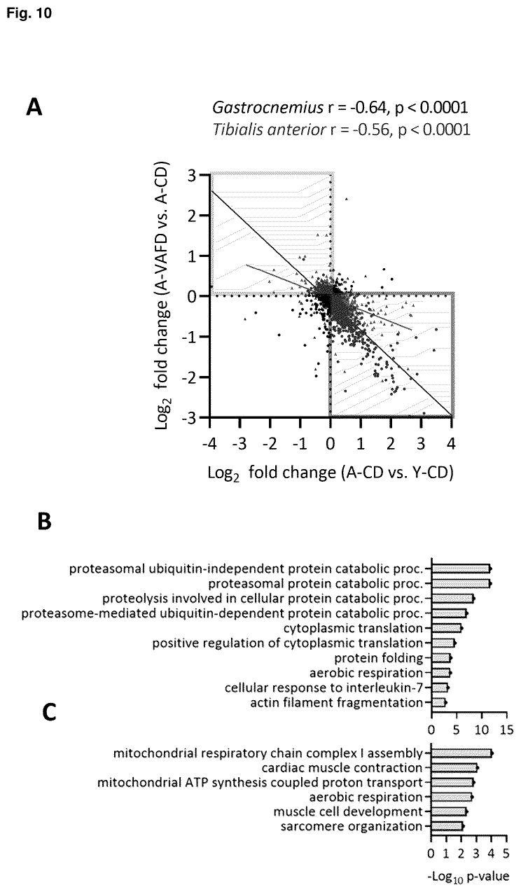

FIG. 10: A, x-y plot of log2 fold changes of protein abundancies determined by mass spectrometry in the tibialis anterior muscle and gastrocnemius muscle of young (10 months) and aged (27 months) mice after receiving a vitamin A control diet (CD, 25 000 IU/kg) or vitamin A free diet (VAFD, <120 IU/kg) for 8 months with facilitated access to food as described in FIG. 4A. Comparison shows A-CD vs. young-CD (x-axis) and A-VAFD vs. A-CD (y-axis). Solid line indicates linear regression (n=5 for all groups). B, Gene ontology analysis (biological processes) of proteins that are up-regulated in both muscle types during aging (A-CD vs. young-CD) but in aged mice that received a VAFD compared to a CD diet (A-VAFD vs. A-CD), (A). C, Gene ontology analysis (biological processes) of proteins that are down-regulated in both muscle types during aging (A-CD vs. young-CD) but up-regulated in aged mice that received a VAFD compared to a CD diet (A-VAFD vs. A-CD), (A). Statistics: Data is represented as mean values. Statistical significance was calculated using Fisher's Exact test with corrections for multiple testing (B, C).

### EXAMPLES

### Dietary Increase of Vitamin A (VA) Supplementation Leads to Reduction Muscle Fiber Size in Skeletal Muscle of Aging Mice

During aging, FAP of skeletal muscle exhibit increases in ROL metabolism (FIG. 1). To address the question whether increases in dietary VA would influence muscle maintenance, cohorts of young adult mice (2 months old) were exposed to a diet containing high concentration of VA (333,000 IU/kg) or a control diet (25,000 IU/kg of VA). Analysis of muscle fibers in the tibialis anterior (TA) muscle of 14-month-old mice of these cohorts, revealed a significant reduction in muscle fiber size (FIG. 3C). This reduction predominantly affected type-2x and type-2b muscle fibers (FIG. 3C). The latter fiber type is known to be most strongly affected during aging-associated muscle atrophy (Miljkovic et al., 2015). However, normalized to the total body weight, there was no significant reduction in muscle weight in the VA high diet vs. control groups (FIG. 3B), possibly due to the overall reduction in body weight and the lean body status observed in VA-high diet group (FIG. 3D). Similar results on VA-high diet induced decrease in muscle fiber size were also observed in regenerated muscle, 10 days after experimentally induced muscle injury of mice of the same cohorts (FIG. 3 E, F).

### Dietary Depletion of Vitamin-A (VA) Ameliorates Aging-Associated Impairments in Skeletal Muscle Function

To determine possible influences of VA on aging muscle, cohorts of young (2 months) and aged mice (19 month) were exposed for 8 months to a VA-free diet (VAFD, VA content <120 IU/kg food) or to a control diet (CD) containing normal levels of VA (CD, VA content =25,000 IU/kg food, FIG. 4A). Since these experimental chows had an increased hardness compared to the normal chow of the mouse house, the chow was provided via the rack (on top of the cage) as well as in the bedding (at the bottom) to avoid a reduction in food uptake and body weight, which was achieved by this measure (FIG. 5A, B). Of note, the Dietary VA-content had no significant effects on body weight or survival of the mice (FIG. 5A-C) .

As expected from literature, measurement of muscle strength at the end of the diet treatment revealed a significant reduction of muscle function in aged mice (27 month old) vs. young mice (10 month old) that were exposed to the CD (FIG. 4B). This was accompanied by a reduction in muscle mass of CD-fed aged vs. young mice (FIG. 4C) and as expected from literature (Harari et al., 2020), a reduction of type 2b myofiber size (FIG. 5D). Importantly, VAFD rescued the aging-associated decline of muscle strength (FIG. 4B). However, the aging-associated decline in muscle mass and myofiber size was not rescued by VAFD (FIG. 4C, FIG. 5D). Together, these data indicated that muscle function declines in healthy aging laboratory mice in association with impairments in maintenance of muscle mass. However, an improvement of muscle function by the VAFD does not necessarily require an increase of muscle mass.

A second experiment was conducted combining aging with a reduction in food intake. To test whether reduction in food intake would aggravate aging-associated loss in the maintenance of skeletal muscle mass, the hardened experimental chow (same as in the above experiment) was given only via the rack (on the top of the cage) without placing extra pieces of chow in the bedding (FIG. 4D). As expected, this protocol led to a reduction in food uptake by 20% (FIG. 5E) accompanied by a 15% reduction in body weight within the first 2 weeks after diet treatment (FIG. 5F). As in the first experiment, aged mice exposed to the experimental diets revealed no difference in survival or body weight (FIG. 5 A, B, C, F, G) indicating that VAFD did not induce toxicity or VA deficiency under these conditions. Six months after initiation of the dietary treatments, CD-fed aged mice exhibited a reduction in tibialis anterior (TA) muscle weight compared to young mice with ad libitum access to standard chow (normal hardness, no reduction in food uptake (FIG. 4E). Under this experimental condition, the reduction in muscle mass between aged mice and young mice appeared to be even more prominent (85% reduction compared to 75% reduction in the first experiment (FIG. 4F), suggesting that a mild reduction in food intake may aggravate age related muscle loss in mice. Importantly, VAFD significantly rescued the loss of muscle weight in aged mice exposed to this protocol of mild DR (FIG. 4E). This improvement of muscle weight in VAFD-treated, aged mice was not associated with changes in overall body weight (FIG. 5H). The relative muscle weight of VAFD-fed, aged mice remained reduced compared to young animals FIG. 4. However, VAFD-fed aged mice with a mild DR were able maintain their muscle mass to a similar extend as VAFD-fed aged mice without DR (FIG. 4F). Similarly, the VAFD feeding to aged mice ameliorated the aging-associated reduction of type 2b myofiber size (FIG. 5I). Together, these results imply that VAFD rescues aging-associated impairments in muscle maintenance, especially those induced by reduction in food intake.

### Dietary Depletion of Vitamin-A (VAFD) does not Change VA Status of Blood, Muscle and Eyes Retinoic Acid (RA) Signaling in Skeletal Muscle

The above analysis of VAFD vs. CD treated mice did not show a diet dependent reduction of retinol in blood serum (FIG. 6A). To determine whether VAFD induced improvements of aged skeletal muscle could be directly linked to VA levels, retinol metabolites and retinoic acid (RA) signaling was measured in skeletal muscle biopsies of the dietary groups. Retinyl-ester (RE), the storage form of VA, was elevated in the muscle of aged vs. young mice (FIG. 6B), but there was no age-dependent difference of retinol and RA content (FIG. 6C, D). To determine possible changes in RA-signaling, the mRNA expression of the prominent RA target genes Rarb and Cyp26a1/b1 in full muscle biopsies, muscle stem cells (MuSCs) and fibro-adipogenic progenitors (FAPs) of CD and VAFD fed mice was analyzed. In none of the sample groups, a difference in Rarb or Cyp26a1/b1 was detected between VAFD and the control diet (FIG. 6 E-G). The function of eyes and visual perception relies on constant levels of VA. Similar to skeletal muscle the measurement of total VA and the essential VA metabolite 11-cis-retinal (11-cis-RAL), which is essential for vision, did not show a drop of these metabolites (FIG. 6 H, I). Of note, slightly elevated levels of total VA in eyes of aged compared to young mice were normalized by the VAFD (FIG. 6 H).

### VAFD Mobilizes Hepatic VA Storages and Rescues Aging-Associated Impairments in Liver Fat Metabolism

In line with the observation that serum retinol and muscular retinol and RA levels remained stable over 6-8 month of VAFD vs. CD feeding of aged mice (FIG. 6)), there was a significant reduction in liver retinol levels in VAFD-treated vs. control fed mice during this period (FIG. 7A). Moreover, a strong reduction of mRNA expression of Cyp26a1 was observed in livers of VAFD vs. CD fed mice (FIG. 7B). These data indicated that the mobilization of hepatic VA storages contributed to maintenance of retinol levels in serum and RA-signaling in muscle. To test whether the mobilization of hepatic VA-storages may induce changes in the liver of aged mice that could contribute to the prevention of aging-associated sarcopenia, a proteome analysis was conducted on liver samples from (i) aged vs. young mice that were exposed to CD and (ii) aged mice that were fed for 8 months with VAFD vs. CD (same experimental cohorts as described in FIG. 4A). Interestingly, there was a strong negative correlation between these two comparisons indicating that the VAFD reversed aging related changes in liver (FIG. 7C). GO-term enrichment analysis revealed that the main pathways that were upregulated with aging and reverted by VAFD were related to processing, splicing, and transport of mRNA (FIG. 7D), whereas the main pathways that were downregulated in aging liver but reactivated by VAFD were related to fat metabolism (FIG. 7E). This latter finding was interesting, since impairments in liver fat metabolism have previously been linked to sarcopenia development, especially in patients with liver diseases, such as cirrhosis, non-alcoholic steatohepatitis (NASH) or non-alcoholic fatty liver disease (NAFLD) (Montano-Loza et al., 2014; Lee et al., 2016; Pasmans et al., 2021). To validate differences in fat metabolism in aging, a targeted metabolomics analysis was conducted using liver biopsies from the same cohorts of mice that were investigated by proteomics analysis. The most consistent difference in the metabolome of aged vs. young, CD-fed mice was a strong increase of fatty acids (PUFAs) (FIG. 7F, FIG. 8)). Importantly, this accumulation of fatty acids in aged liver was almost completely rescued by VAFD-feeding. Histological and biochemical analysis confirmed a significant reduction of lipid accumulation in the livers VAFD vs. CD fed mice (FIG. 7G, H). Interestingly, also triglyceride levels in blood plasma samples of VAFD vs. CD fed mice were reduced (FIG. 7I). Additionally, a first trial with 3 volunteers revealed that 6 weeks of a VAFD were able to reduce total and LDL-cholesterin levels in the blood compared to the starting point of the experiment. Blood levels of Vitamin A exhibited a slight drop for 2 out of 3 participants but remained in the recommended physiological range (FIG. 7J). Together, these findings indicate that VAFD rescues aging related impairments of fatty acid metabolism in the liver—a condition, which is also known to be associated with the development of sarcopenia.

### VAFD-Mediated Rescue in Fat Metabolism of Aged Liver Involve Pparα/Fgf21—Activation and Chromatin Silencing of Fat Metabolism Suppressive Pathways

An upstream regulator analysis (IPA, Qiagen) of differentially expressed proteins (DEPs) was conducted on DEPs that were downregulated in liver of aged vs. young mice but rescued in aged mice that were exposed to VAFD vs. CD (same experiment as shown in FIG. 7C, upper left quadrant). This analysis revealed PPARα as the strongest candidate involved in the upregulation of DEPs in liver of aged mice exposed to VAFD (FIG. 9A). PPARα is known as major regulator for the activation of hepatic lipid metabolism. In contrast, the strongest candidate involved in the downregulation of DEPs in liver of aged mice exposed to VAFD vs. CD was Slc27a2, also known as Fatp2—a regulator of FA transport (FIG. 9A). Slc27a2 is known to be upregulated in liver diseases of impaired fat metabolism (NASH, NAFLD). To corroborate our result on the potentially increased PPARα-activity in the liver of aged mice that were exposed to VAFD vs. CD, we plotted the log2 fold changes of proteins that are encoded by known PPARα target genes (Rakhshandehroo et al., 2010). In confirmation of the above results, PPARα targets were predominantly downregulated in aged vs. young mice that were fed with the control diet (FIG. 9B) but upregulated in aged VAFD-fed vs. CD-fed mice (FIG. 9C). Currently the agonist of the glucagon-like peptide-1 receptor (GLP1-RA) is considered one of the best candidates for pharmacological treatment of fatty liver diseases (Nevola et al., 2023). Notably, the treatment of mice that have a diet induced nonalcoholic steatohepatitis (NASH) with GLP1-RA showed an induction of PPARa target genes compared to the control group (NaCl) control that was similar or even slightly lower that the effect of the aged VAFD-fed vs. CD-fed mice (FIG. 9D).

FGF21 is a target gene of PPARα (Lundasen et al., 2007). In response to starvation, FGF21 is predominantly produced by the liver and is thus referred to as a hepatokine. Hepatic FGF21 activates fat metabolism in liver as well as glucose uptake and metabolic activity of peripheral organs including skeletal muscle (Flippo et al., 2021; Sun et al., 2021). Of note, FGF21 was upregulated in VAFD-fed vs. CD-fed mice at both young and old age (FIG. 9E). These data suggested that PPARα/FGF21 induction may contribute to the improvements in liver fat metabolism as well as in improvements in muscle maintenance and function of aged, VAFD treated mice. Additionally, we observed an increase of serum beta hydroxy butyrate (BHB) levels in aged mice that were treated with the VAFD (FIG. 9F). BHB is a keton body that originated from the metabolism of fatty acids in the liver and has shown to be positive effects on aging associated functional declines (Han et al., 2020).

Together, the above experiments provided experimental evidence that PPARα/FGF21 activation contributes to the improvement of liver fat metabolism of aged mice in response to VAFD-treatment. PPARα is known to activate fat metabolism by increasing the expression of genes involved in the beta oxidation of fatty acids (Wang et al., 2020; Lundasen et al., 2007; Tahri-Joutey et al., 2021). In addition, PPARα has also been shown to downregulate the activity of pathways that are known to inhibit fat metabolism in aging and/or inflamed liver, such as NFκB and TNFα signaling (Bougarne et al., 2018). Inflammatory pathways are regulated in an inducible manner strongly depending on co-stimulatory processes involving for example circadian regulation, individual timing of food uptake, and possibly also circuit among different cell types of the liver. Following this reasoning, RNA sequencing experiments and the analysis of bulk tissue biopsies have limitations in assessing the possible regulation of such pathways. To determine whether changes in chromatin openness—a more stable marker of gene regulatory changes than RNAseq—would occur in different cell types of aged mice that were exposed to CD or VAFD, a single-nucleus ATAC-sequencing experiment was conducted on freshly isolated liver nuclei. Principle component analysis of the accessibility of chromatin regions revealed a clear separation of clustering of different cell types of the liver (FIG. 9G). Interestingly, VAFD compared to CD induced a strong shift of the hepatocyte fraction of liver nuclei (FIG. 9G). Next, a list of genes that showed an enrichment or depletion of accessibility peaks in the vicinity of their promotes to the Ingenuity pathway analysis tool (IPA, Qiagen) was submitted and the upstream regulator prediction was used to identify potential regulators of the genes with the observed chromatin changes. The analysis revealed that many of the top 10 ranked upstream regulators whose activity was predicted to be reduced in hepatocytes of old VAFD-vs CD-fed mice are known to have a prominent role in suppressing liver fat metabolism including NFκB signaling (Liang et al., 2014), TNFα signaling (Wandrer et al., 2020), SOX4, TWIST1, NROB2, IFNG and AGT (FIG. 9H). In agreement with the upstream regulator analysis that was conducted on differentially expressed proteins in livers of VAFD-fed vs. CD-fed mice (FIG. 8A), also chromatin changes in hepatocytes of the same mice predict an enhanced activity of PPARα (FIG. 9H) These data confirm on chromatin level that VAFD-fed vs CD-fed old mice possess an enhanced liver metabolism, which is predominantly originating from hepatocytes. Moreover, the data suggests that a VAFD might ameliorate aging associated increases of hepatic inflammation.

### VAFD Ameliorates Metabolic Compromise, Protein Catabolismand Maintenance of Structural Proteins of Muscle Contraction in Aging Muscle

To gain insights into the molecular processes involved in the prevention of the development of aging-associated sarcopenia by VAFD treatment of aged mice, a proteomics analysis was conducted on full tissue biopsies of the tibialis anterior (TA) and gastrocnemius (GA) muscles. There was a very strong negative correlation between aging induced changes and changes in skeletal muscle of aged VAFD-fed mice vs. aged CD-fed mice (FIG. 10). GO term analysis revealed that the main biological processes that were upregulated during aging and reverted by VAFD were related to protein catabolismcompensatory increases in translation, and increases in inflammation signaling (FIG. 10B). In contrast, GO term analysis of biological processes on proteins that were downregulated in aging but increased by the VAFD in aged muscles showed a significant enrichment processes related to muscle contraction and energy metabolism (FIG. 10C). Together, these data indicate that VAFD has the potential to revert major molecular and metabolic processes, which are known to be causatively related to sarcopenia (Campos et al., 2018; Altun et al., 2010; Kimball et al., 2004; Rieu et al., 2009; Narici et al., 2021; Migliavacca et al., 2019).

Overall, it is shown that late life treatment of aged mice with VAFD represents a powerful dietary intervention, which prevents aging-related impairments in skeletal muscle mass and function in aged mice. Dietary interventions that have a comparable efficiency in sarcopenia prevention are currently not available but were long sought after. Importantly, VAFD-treatment of aged mice has no side effects since aged mice have sufficient VA storages to fully compensate for dietary VA depletion by keeping constant levels of serum retinol. In this way, laboratory mice reflect the situation in the aged human population of western countries showing high levels of dietary VA, high storage levels of VA, and a very rare occurrence of VA deficiencies (Zhao et al., 2022; Mensink et al., 2013). Mechanistically, the study shows that VAFD does not directly affect RA signaling in muscle. Instead, VAFD leads to a strong rescue of aging-associated impairments in liver fat metabolism—a condition that has been linked to the development of sarcopenia in patients with liver diseases that are characterized by impaired fat metabolism, such as cirrhosis, NAFLD and NASH (De Bandt et al., 2018). The findings provide a proof of concept that the reversal of aging-associated impairments in liver fat metabolism ameliorate the development of sarcopenia. It is shown that the reduction of PPARα signaling contributes to aging-associated impairments in liver fat metabolism, which however, is strongly reverted by VAFD treatment of aged mice. It is possible that the release of RA in liver directly contributes to activation PPAR signaling. In addition, PPAR signaling might be enhanced by the corelease of long-chain fatty acids (known activators of PPAR) during mobilization of retinylesters. Finally, VAFD mediated effects on the intestinal microbiome composition contribute to the activation of PPAR signaling and chromatin modification thereby contributing to the reactivation of liver fat metabolism.

### REFERENCES

1. Rosenberg, Irwin H. “Sarcopenia: origins and clinical relevance.” The Journal of nutrition 127.5 (1997): 990S-991S.
2. Cruz-Jentoft, Alfonso J., et al. “Sarcopenia: revised European consensus on definition and diagnosis.” Age and ageing 48.1 (2019 ): 16-31.
3. Lo, J, et al. “Sarcopenia: Current treatments and new regenerative therapeutic approaches.” Journal of orthopaedic translation 23 (2020): 38-52.
4. Wu, P Y, et al. “Exercise, Nutrition, and combined exercise and nutrition in older adults with sarcopenia: A systematic review and network meta-analysis.” Maturitas 145 (2021): 38-48.
5. Pietrocola, F., et al. “Acetyl coenzyme A: a central metabolite and second messenger.” Cell metabolism 21.6 (2015 ): 805-821.
6. Dhaliwal, Amritpal, and Matthew J. Armstrong. “Sarcopenia in cirrhosis: A practical overview.” Clinical Medicine 20.5 (2020 ): 489.
7. Lee, Jieun, et al. “Hepatic fatty acid oxidation restrains systemic catabolismduring starvation.” Cell reports 16.1 (2016 ): 201-212.
8. Cariou, Bertrand. “The metabolic triad of non-alcoholic fatty liver disease, visceral adiposity and type 2 diabetes: Implications for treatment.” Diabetes, Obesity and Metabolism 24 (2022): 15-27.
9. Rinaldi, Luca, et al. “Mechanisms of non-alcoholic fatty liver disease in the metabolic syndrome. A narrative review.” Antioxidants 10.2 (2021 ): 270.
10. Halle, M, et al. Meal replacement by formula diet reduces weight more than a lifestyle intervention alone in patients with overweight or obesity and accompanied cardiovascular risk factors-the ACOORH trial. Eur J Clin Nutr. 2021; 75:661-669.
11. Kelmenson, D A, et al. Outcomes of ICU Patients With a Discharge Diagnosis of Critical Illness Polyneuromyopathy: A Propensity-Matched Analysis. Crit Care Med. 2017; 45:2055-60.
12. Fleischmann-Struzek et al. Hospitalization and Intensive Therapy at the End of Life. A national analysis of DRG statistics from 2007-2015 Dtsch Arztebl Int 2019; 116:653-60.
13. Reinhold S, et al. Protein delivery in intermittent and continuous enteral nutrition with a protein-rich formula in critically ill patients-a protocol for the prospective randomized controlled proof-of-concept Protein Bolus Nutrition (Pro BoNo) study. Trials. 2020; 21:740.
14. Beckwe, David, et al. “Exercise interventions for the prevention and treatment of sarcopenia. A systematic umbrella review.” The journal of nutrition, health & aging 23.6 (2019 ): 494-502.
15. Peterson, M. D., A. Sen, and P. M. Gordon, Influence of resistance exercise an lean body mass in aging adults: a meta-analysis. Med Sci Sports Exerc, 2011; 43 (2): p. 249-58.
16. Peterson, M. D., et al., Resistance exercise for muscular strength in older adults: A metaanalysis. Ageing Research Reviews, 2010; 9 (3): p. 226-237. Becker, et al., 2015;
17. Woodhouse L, Gandhi R, Warden S J, Poiraudeau S, Myers S L, Benson C T, et al. A phase 2 randomized study investigating the efficacy and safety of myostatin antibody LY2495655 versus placebo in patients undergoing elective total hip arthroplasty. J Frailty Aging. 2016; 5:62-70. Rook, et al. 2017,
18. Campbell C, McMillan H J, Mah J K, et al. Myostatin inhibitor ACE-031 treatment of ambulatory boys with Duchenne muscular dystrophy: Results of a randomized, placebo-controlled clinical trial. Muscle Nerve. 2017; 55:458-464. Kwak, et al. 2019
19. Sinha-Hikim I, Cornford M, Gaytan H, Lee M L, Bhasin S (2006) Effects of testosterone supplementation on skeletal muscle fiber hypertrophy and satellite cells in community-dwelling older men. J Clin Endocrinol Metab 91:3024-3033 Malafarina, et al., 2012
20. Skinner, Jared W., et al. “Muscular responses to testosterone replacement vary by administration route: a systematic review and meta-analysis.” Journal of cachexia, sarcopenia and muscle 9.3 (2018 ): 465-481
21. Dayal M, Sammel M D, Zhao J, Hummel A C, Vandenbourne K, Barnhart K T (2005) Supplementation with DHEA: effect on muscle size, strength, quality of life, and lipids. J Women's Health 14:391-400
22. Dalton J T, Barnette K G, Bohl C E, Hancock M L, Rodriguez D, Dodson S T, et al. The selective androgen receptor modulator GTx-024 (enobosarm) improves lean body mass and physical function in healthy elderly men and postmenopausal women: results of a double-blind, placebo-controlled phase II trial. J Cachexia Sarcopenia Muscle. 2011; 2:153-61
23. Papanicolaou D A, Ather S N, Zhu H, Zhou Y, Lutkiewicz J, Scott B B, et al. A phase HA randomized, placebo-controlled clinical trial to study the efficacy and safety of the selective androgen receptor modulator (SARM), MK-0773 in female participants with sarcopenia. J Nutr Health Aging. 2013; 17:533-
23. Nagaya N, Moriya J, Yasumura Y, Uematsu M, Ono F, Shimizu W, Ueno K, Kitakaze M, Miyatake K, Kangawa K (2004) Effects of ghrelin administration on left ventricular function, exercise capacity, and muscle wasting in patients with chronic heart failure. Circulation
25. Temel J S, Abernethy A P, Currow D C, Friend J, Duus E M, Yan Y, Fearon K C (2016) Anamorelin in patients with non-small-cell lung cancer and cachexia (ROMANA 1 and ROMANA 2): results from two randomized, double-blind, phase 3 trials. Lancet Oncol 17:519-531
26. Dickinson J M, Reidy P T, Gundermann D M, Borack M S, Walker D K, D'Lugos AC, Volpi E, Rasmussen B B (2017) The impact of postexercise essential amino acid ingestion an the ubiquitin proteasome and autophagosomal-lysosomal systems in skeletal muscle of older men. J Appl Physiol 122:620-630
27. Walker D K, Dickinson J M, Timmerman K L, Drummond M J, Reidy P T, Fry C S, Gundermann D M, Rasmussen B B (2011) Exercise, amino acids, and aging in the control of human muscle protein synthesis. Med Sci Sports Exerc 43:2249-2258
28. Dillon E L, Sheffield-Moore M, Paddon-Jones D, Gilkison C, Sanford A P, Casperson S L, Jiang J, Chinkes D L, Urban R J (2009) Amino acid supplementation increases lean body mass, basal muscle protein synthesis, and insulin-like growth factor-I expression in older women. J Clin Endocrinol Metab 94:1630-1637
29. Esmarck B, Andersen J L, Olsen S, Richter E A, Mizuno M, Kjaer M (2005) Timing of postexercise protein intake is important for muscle hypertrophy with resistance training in elderly humans. J Physiol 535:301-311
30. Godard M P, Williamson D L, Trappe S W (2002) Oral amino-acid provision does not affect muscle strength or size gains in older men. Med Sci Sports Exerc 34:1126-1131
31. M. Rondanelli, C. Klersy, G. Terracol, J. Talluri, R. Maugeri, D. Guido, M. A. Faliva, B. S. Solerte, M. Fioravanti, H. Lukaski, S. Perna, Whey protein, amino acids, and vitamin D supplementation with physical activity increases fat-free mass and strength, functionality, and quality of life and decreases inflammation in sarcopenic elderly. Am. J. Clin. Nutr. 103, 830-840 (2016)
32. A. M. Verreijen, S. Verlaan, M. F. Engberink, S. Swinkels, J. de Vogel-van den Bosch, P. J. Weijs, A high whey protein-, leucine-, and vitamin D-enriched supplement preserves muscle mass during intentional weight loss in obese older adults: A double-blind randomized controlled trial-. Am. J. Clin. Nutr. 101, 279-286 (2014)
33. Latham, N. K.; Anderson, C. S.; Lee, A.; Bennett, D. A.; Moseley, A.; Cameron, I. D. A randomized, controlled trial of quadriceps resistance exercise and vitamin D in frail older people: The Frailty Interventions Trial in Elderly Subjects (FITNESS). J. Am. Geriatr. Soc. 2003, 51,291-299.
34. Shea, M. K.; Fielding, R. A.; Dawson-Hughes, B. The effect of vitamin D supplementation on lower-extremity power and function in older adults: A randomized controlled trial. Am. J. Clin. Nutr. 2019, 10, 1093.
35. Levis, S.; Gomez-marin, 0. Vitamin D and Physical Function in Sedentary Older Men. JAGS 2016, 65, 323-331
36. Ferguson, Daniel, and Brian N. Finck. “Emerging therapeutic approaches for the treatment of NAFLD and type 2 diabetes mellitus.” Nature Reviews Endocrinology 17.8 (2021 ): 484-495. Miljkovic et al., 2015
37. Gross, Barbara, et al. “PPARs in obesity-induced T2DM, dyslipidemia and NAFLD.” Nature Reviews Endocrinology 13.1 (2017 ): 36-49.
38. Brandi, Giovanni, et al. “Microbiota, NASH, HCC and the potential role of probiotics.” Carcinogenesis 38.3 (2017 ): 231-240.
39. Harari, Ayelet, et al. “Dietary β-carotene rescues vitamin A deficiency and inhibits atherogenesis in apolipoprotein E-deficient mice.” Nutrients 12.6 (2020 ): 1625.
40. Cruz-Jentoft, Alfonso J., et al. “Nutrition, frailty, and sarcopenia.” Aging clinical and experimental research 29.1 (2017 ): 43-48.
41. Wang, Yaping, et al. “PPARs as metabolic regulators in the liver: lessons from liver-specific PPAR-null mice.” International journal of molecular sciences 21.6 (2020 ): 2061.
42. Rakhshandehroo, Maryam, et al. “Peroxisome proliferator-activated receptor alpha target genes.” PPAR research 2010 (2010).
43. Nevola, Riccardo, et al. “GLP-1 receptor agonists in non-alcoholic fatty liver disease: current evidence and future perspectives.” International Journal of Molecular Sciences 24.2 (2023 ): 1703.
44. Lundåsen, Thomas, et al. “PPARα is a key regulator of hepatic FGF21.” Biochemical and biophysical research communications 360.2 (2007 ): 437-440.
45. Flippo, Kyle H., and Matthew J. Potthoff. “Metabolic messengers: FGF21.” Nature metabolism 3.3 (2021 ): 309-317.
46. Sun, Hui, Matthew Sherrier, and Hongshuai Li. “Skeletal Muscle and Bone-Emerging Targets of Fibroblast Growth Factor-21.” Frontiers in Physiology (2021): 269.
47. Tahri-Joutey, Mounia, et al. “Mechanisms mediating the regulation of peroxisomal fatty acid beta-oxidation by PPARα.” International Journal of Molecular Sciences 22.16 (2021 ):8969.
48. Han, Young-Min, Tharmarajan Ramprasath, and Ming-Hui Zou. “β-hydroxybutyrate and its metabolic effects on age-associated pathology.” Experimental & Molecular Medicine 52.4 (2020 ): 548-555.
49. Bougarne, Nadia, et al. “Molecular actions of PPARα in lipid metabolism and inflammation.” Endocrine reviews 39.5 (2018 ): 760-802.
50. Liang, Wen, et al. “Metabolically induced liver inflammation leads to NASH and differs from LPS-or IL-1β-induced chronic inflammation.” Laboratory investigation 94.5 (2014 ): 491-502.
51. Wandrer, Franziska, et al. “TNF-Receptor-1 inhibition reduces liver steatosis, hepatocellular injury and fibrosis in NAFLD mice.” Cell Death & Disease 11.3 (2020 ): 1-9.
52. Jiao, Yang, et al. “SRY-box containing gene 4 promotes liver steatosis by upregulation of SREBP-1c.” Diabetes 67.11 (2018 ): 2227-2238.
53. Yang, Xiaoxue, et al. “Twist1-induced miR-199a-3p promotes liver fibrosis by suppressing caveolin-2 and activating TGF-β pathway.” Signal transduction and targeted therapy 5.1 (2020 ): 1-12.
54. Huang, Jiansheng, et al. “Molecular characterization of the role of orphan receptor small heterodimer partner in development of fatty liver.” Hepatology 46.1 (2007 ): 147-157.
55. Luo, Xiao-Yu, et al. “IFN-γ deficiency attenuates hepatic inflammation and fibrosis in a steatohepatitis model induced by a methionine-and choline-deficient high-fat diet.” American Journal of Physiology-Gastrointestinal and Liver Physiology 305.12 (2013 ): G891-G899
56. Georgescu, Eugen Florin, et al. “Angiotensin-receptor blockers as therapy for mild-to-moderate hypertension-associated non-alcoholic steatohepatitis.” World journal of gastroenterology: WJG 15.8 (2009 ): 942.
57. De Bandt, Jean-Pascal, Prasanthi Jegatheesan, and Naouel Tennoune-El-Hafaia. “Muscle loss in chronic liver diseases: the example of nonalcoholic liver disease.” Nutrients 10.9 (2018 ): 1195.
58. Campos, Fabián, et al. “Sarcopenia in a mice model of chronic liver disease: role of the ubiquitin-proteasome system and oxidative stress.” Pflügers Archiv-European Journal of Physiology 470.10 (2018 ): 1503-1519.
59. Altun, Mikael, et al. “Muscle wasting in aged, sarcopenic rats is associated with enhanced activity of the ubiquitin proteasome pathway.” Journal of Biological Chemistry 285.51 (2010 ): 39597-39608.
60. Kimball, Scot R., et al. “Assessment of biomarkers of protein anabolism in skeletal muscle during the life span of the rat: sarcopenia despite elevated protein synthesis.” American Journal of Physiology-Endocrinology and Metabolism 287.4 (2004 ): E772-E780.
61. Rieu, Isabelle, et al. “Reduction of low grade inflammation restores blunting of postprandial muscle anabolism and limits sarcopenia in old rats.” The Journal of physiology 587.22 (2009 ): 5483-5492.
62. Narici, Marco, et al. “Age-related alterations in muscle architecture are a signature of sarcopenia: the ultrasound sarcopenia index.” Journal of Cachexia, Sarcopenia and Muscle 12.4 (2021): 973-982.
63. Migliavacca, Eugenia, et al. “Mitochondrial oxidative capacity and NAD+biosynthesis are reduced in human sarcopenia across ethnicities.” Nature communications 10.1 (2019 ): 1-14.
64. Zhao, Tian, et al. “Global burden of vitamin A deficiency in 204 countries and territories from 1990-2019.” Nutrients 14.5 (2022 ): 950.
65. Mensink, G. B. M., et al. “Mapping low intake of micronutrients across Europe.” British journal of nutrition 110.4 (2013 ): 755-773.
66. Miljkovic, Natasa, et al. “Aging of skeletal muscle fibers.” Annals of rehabilitation medicine 39.2 (2015 ): 155-162.

1. A method for reducing the risk of and/or treating an impairment and/or dysfunction of liver fat metabolism and/or a medical condition associated therewith, comprising administering a composition as a dietary treatment to a subject in need thereof, wherein the composition comprises dietary nutrients and is substantially free of vitamin A and/or derivatives thereof
wherein administering said composition reduces the risk of and/or treats an impairment and/or dysfunction of liver fat metabolism and/or a medical condition associated therewith.

2. The method according to claim 1, wherein the composition comprises an amount of dietary nutrients covering the daily requirements, or a part thereof, of a subject.

3. The method according to claim 1, wherein the composition comprises a processed food product.

4. The method according to claim 1, wherein the dietary nutrients comprise carbohydrates, proteins, fats, vitamins, minerals, trace elements, secondary plant substances, derivatives and/or chemical precursors thereof and/or probiotic substances.

5. The method according to claim 1, wherein a total amount of vitamin A or derivatives thereof received by the subject is less than 5% of the daily requirement of Vitamin A or derivatives thereof of a subject.

6. The method according to claim 1, wherein a total amount of vitamin A or derivatives thereof received by the subject is less than 50 μg per day.

7. The method according to claim 1, wherein the dietary treatment comprises replacing the diet of the subject or parts thereof by the composition.

8. The method according to claim 1, wherein the composition is configured to be administered periodically, selected from the list consisting of at least once weekly, at least once daily, and two or more times daily.

9. The method according to claim 1, wherein the composition is configured to be administered orally, enterally, or parenterally to the subject.

10. The method according to claim 1, wherein the medical condition associated with an impairment and/or dysfunction of liver fat metabolism is a liver disease.

11. The method according to claim 1, wherein the medical condition associated with an impairment and/or dysfunction of liver fat metabolism is a metabolic disease.

12. The method according to claim 1, wherein the medical condition associated with an impairment and/or dysfunction of liver fat metabolism is a muscle disease.

13. The method according to claim 1, wherein the medical condition associated with an impairment and/or dysfunction of liver fat metabolism is present in a subject ≥30 years.

14. The method according to claim 12, wherein the muscle disease is associated with reduced mobility of the subject.

15. The method according to claim 1, wherein the impairment and/or dysfunction of liver fat metabolism is associated with one or more conditions selected from the list consisting of an intensive care intervention, intensive care unit-acquired weakness (ICU-AW), hospitalization, a prolonged period of reduced mobility, a prolonged bed-ridden condition, perioperative loss of muscle mass and/or muscle strength, immobilization of injured body parts and/or loss of motor neuron function.

16. The method according to claim 1, wherein the impairment and/or dysfunction of liver fat metabolism is associated with a reduced and/or altered food intake and/or malnutrition.

17. The method according to claim 1, wherein the impairment and/or dysfunction of liver fat metabolism is associated with one or more conditions selected from the list consisting of cachexia caused by cancer, age of the subject, a genetic predisposition of the subject to impairments in liver fat metabolism, rheumatoid arthritis, chronic renal insufficiency, chronic heart failure, acquired immune deficiency syndrome (AIDS), chronic lung diseases with respiratory insufficiency, chronic obstructive pulmonary disease (COPD) and/or diabetes mellitus type I.

18. The method according to claim 5, wherein the total amount of vitamin A or derivatives thereof received by the subject is less than 2.5% of the daily requirement thereof and/or less than 25 μg per day, and the dietary treatment comprises fully replacing the diet of the subject or parts thereof by the composition.

19. The method according to claim 1, wherein the subject is ≥40 years of age.

20. The method according to claim 1, wherein the subject is ≥60 years of age.
